SEQUENCE LISTING (14 sequences)

SEQ ID NO: 1 - DNA, 546 bp, Homo sapiens
  CDS (1)..(546)
atgatgcttcgggtcctggtgggggctgtcctccctgccatgctactggctgccccaccacccatcaacaagctggcactgttcccagataagagtgcctggtgcgaagccaagaacatcacccagatcgtgggccacagcggctgtgaggccaagtccatccagaacagggcgtgcctaggacagtgcttcagctacagcgtccccaacaccttcccacagtccacagagtccctggttcactgtgactcctgcatgccagcccagtccatgtgggagattgtgacgctggagtgcccgggccacgaggaggtgcccagggtggacaagctggtggagaagatcctgcactgtagctgccaggcctgcggcaaggagcctagtcacgaggggctgagcgtctatgtgcagggcgaggacgggccgggatcccagcccggcacccaccctcacccccatccccacccccatcctggcgggcagacccctgagcccgaggacccccctggggccccccacacagaggaagagggggctgaggactga

SEQ ID NO: 2 - protein, 181 aa, Homo sapiens
MMLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 3 - DNA, 432 bp, Homo sapiens
  CDS (1)..(432)
atgagccgcacagcctacacggtgggagccctgcttctcctcttggggaccctgctgccggctgctgaagggaaaaagaaagggtcccaaggtgccatccccccgccagacaaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaa

SEQ ID NO: 4 - protein, 143 aa, Homo sapiens
MSRTAYTVGALLLLLGTLLPAAEGKKKGSQGAIPPPDKALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 5 - DNA, 507 bp, Homo sapiens
  CDS (1)..(507)
atgttctggaagctttccctgtccttgttcctggtggcggtgctggtgaaggtggcggaagcccggaagaaccggccggcgggcgccatcccctcgccttacaaggacggcagcagcaacaactcggagagatggcagcaccagatcaaggaggtgctggcctccagccaggaggccctggtggtcaccgagcgcaagtacctcaagagtgactggtgcaagacgcagccgctgcggcagacggtgagcgaggagggctgccggagccgcaccatcctcaaccgcttctgctacggccagtgcaactccttctacatcccgcggcacgtgaagaaggaggaggagtccttccagtcctgcgccttctgcaagccccagcgcgtcacctccgtcctcgtggagctcgagtgccccggcctggacccacccttccgactcaagaaaatccagaaggtgaagcagtgccggtgcatgtccgtgaacctgagcgactcggacaagcagtga

SEQ ID NO: 6 - protein, 168 aa, Homo sapiens
MFWKLSLSLFLVAVLVKVAEARKNRPAGAIPSPYKDGSSNNSERWQHQIKEVLASSQEALVVTERKYLKSDWCKTQPLRQTVSEEGCRSRTILNRFCYGQCNSFYIPRHVKKEEESFQSCAFCKPQRVTSVLVELECPGLDPPFRLKKIQKVKQCRCMSVNLSDSDKQ

SEQ ID NO: 7 - DNA, 804 bp, Homo sapiens
  CDS (1)..(804)
atgcatctcctcttatttcagctgctggtactcctgcctctaggaaagaccacacggcaccaggatggccgccagaatcagagttctctttcccccgtactcctgccaaggaatcaaagagagcttcccacaggcaaccatgaggaagctgaggagaagccagatctgtttgtcgcagtgccacaccttgtagccaccagccctgcaggggaaggccagaggcagagagagaagatgctgtccagatttggcaggttctggaagaagcctgagagagaaatgcatccatccagggactcagatagtgagcccttcccacctgggacccagtccctcatccagccgatagatggaatgaaaatggagaaatctcctcttcgggaagaagccaagaaattctggcaccacttcatgttcagaaaaactccggcttctcagggggtcatcttgcccatcaaaagccatgaagtacattgggagacctgcaggacagtgcccttcagccagactataacccacgaaggctgtgaaaaagtagttgttcagaacaacctttgctttgggaaatgcgggtctgttcattttcctggagccgcgcagcactcccatacctcctgctctcactgtttgcctgccaagttcaccacgatgcacttgccactgaactgcactgaactttcctccgtgatcaaggtggtgatgctggtggaggagtgccagtgcaaggtgaagacggagcatgaagatggacacatcctacatgctggctcccaggattcctttatcccaggagtttcagcttga

SEQ ID NO: 8 - protein, 267 aa, Homo sapiens
MHLLLFQLLVLLPLGKTTRHQDGRQNQSSLSPVLLPRNQRELPTGNHEEAEEKPDLFVAVPHLVATSPAGEGQRQREKMLSRFGRFWKKPEREMHPSRDSDSEPFPPGTQSLIQPIDGMKMEKSPLREEAKKFWHHFMFRKTPASQGVILPIKSHEVHWETCRTVPFSQTITHEGCEKVVVQNNLCFGKCGSVHFPGAAQHSHTSCSHCLPAKFTTMHLPLNCTELSSVIKVVMLVEECQCKVKTEHEDGHILHAGSQDSFIPGVSA

SEQ ID NO: 9 - DNA, 570 bp, Homo sapiens
  CDS (1)..(570)
atgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatga

SEQ ID NO: 10 - protein, 189 aa, Homo sapiens
MLLGQLSTLLCLLSGALPTGSGRPEPQSPRPQSWAAANQTWALGPGALPPLVPASALGSWKAFLGLQKARQLGMGRLQRGQDEVAAVTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKA

SEQ ID NO: 11 - DNA, 642 bp, Homo sapiens
  CDS (1)..(642)
atgcagctcccactggccctgtgtctcgtctgcctgctggtacacacagccttccgtgtagtggagggccaggggtggcaggcgttcaagaatgatgccacggaaatcatccccgagctcggagagtaccccgagcctccaccggagctggagaacaacaagaccatgaaccgggcggagaacggagggcggcctccccaccacccctttgagaccaaagacgtgtccgagtacagctgccgcgagctgcacttcacccgctacgtgaccgatgggccgtgccgcagcgccaagccggtcaccgagctggtgtgctccggccagtgcggcccggcgcgcctgctgcccaacgccatcggccgcggcaagtggtggcgacctagtgggcccgacttccgctgcatccccgaccgctaccgcgcgcagcgcgtgcagctgctgtgtcccggtggtgaggcgccgcgcgcgcgcaaggtgcgcctggtggcctcgtgcaagtgcaagcgcctcacccgcttccacaaccagtcggagctcaaggacttcgggaccgaggccgctcggccgcagaagggccggaagccgcggccccgcgcccggagcgccaaagccaaccaggccgagctggagaacgcctactag

SEQ ID NO: 12 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 13 - DNA, 621 bp, Homo sapiens
  CDS (1)..(621)
atgcttcctcctgccattcatttctatctccttccccttgcatgcatcctaatgaaaagctgtttggcttttaaaaatgatgccacagaaatcctttattcacatgtggttaaacctgttccagcacaccccagcagcaacagcacgttgaatcaagccagaaatggaggcaggcatttcagtaacactggactggatcggaacactcgggttcaagtgggttgccgggaactgcgttccaccaaatacatctctgatggccagtgcaccagcatcagccctctgaaggagctggtgtgtgctggcgagtgcttgcccctgccagtgctccctaactggattggaggaggctatggaacaaagtactggagcaggaggagctcccaggagtggcggtgtgtcaatgacaaaacccgtacccagagaatccagctgcagtgccaagatggcagcacacgcacctacaaaatcacagtagtcactgcctgcaagtgcaagaggtacacccggcagcacaacgagtccagtcacaactttgagagcatgtcacctgccaagccagtccagcatcacagagagcggaaaagagccagcaaatccagcaagcacagcatgagttag

SEQ ID NO: 14 - protein, 206 aa, Homo sapiens
MLPPAIHFYLLPLACILMKSCLAFKNDATEILYSHVVKPVPAHPSSNSTLNQARNGGRHFSNTGLDRNTRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESMSPAKPVQHHRERKRASKSSKHSMS

(19) United States
(12) Patent Application Publication
(10) Pub. No.: US 2022/0265772 A1
(43) Pub. Date: Aug. 25, 2022

(54) USES OF DAN FAMILY BMP ANTAGONISTS FOR INHIBITING OCULAR NEOVASCULARIZATION AND TREATING OCULAR CONDITIONS

(71) Applicant: 9636137 Canada Inc., Rosemère (CA)

(72) Inventors: Gilbert BERNIER, Rosemère (CA); Bruno LARRIVÉE, Montréal (CA)

(73) Assignee: 9636137 Canada Inc., Rosemère, QC (CA)

(21) Appl. No.: 17/630,776

(22) PCT Filed: Jul. 29, 2020
(86) PCT No.: PCT/IB2020/057162

§ 371 Date: Jan. 27, 2022

Related U.S. Application Data
(60) Provisional application No. 62/879,755, filed on Jul. 29, 2019.

(51) Int. Cl. A61K 38/19; A61K 9/00; A61P 27/02; A61K 35/30

(52) U.S. Cl. CPC A61K 38/19; A61K 9/0019; A61P 27/02; A61K 35/30; A61K 9/0048

(57) ABSTRACT

Described herein are methods and pharmaceutical compositions for the prevention and/or treatment of visual impairment and vision loss, and more particularly to the use of DAN family BMP antagonist(s) for the prevention of ocular neovascularization and, as a trophic factor for photoreceptors in eye diseases. Also described is a method for long-term inhibition of neovascularization in an ocular condition, a method of replacement therapy for vascular endothelial growth factor (VEGF) inhibitor treatment and a method for inhibiting and/or preventing ocular neovascularization and/or ocular angiogenesis in a mammalian subject, the methods comprising administering to a mammalian subject in need an effective amount a DAN family BMP antagonist such as DAND5.

### FIELD OF THE INVENTION

This invention relates to the field of visual impairment and vision loss, and more particularly to the use of DAN family BMP antagonist(s) and methods thereof for, first, the prevention of ocular neovascularization and, second, as a trophic factor for photoreceptors in eyes diseases.

### BACKGROUND OF THE INVENTION

Ocular neovascularization is a common central factor to several ocular diseases leading to visual impairment and vision loss, including diseases such as proliferative diabetic retinopathy, retinopathy of prematurity, and neovascular age-related macular degeneration (AMD).

For example, age-related macular degeneration (AMD) is one of the leading causes of blindness worldwide, exceeded by glaucoma and cataract only. In the elderly, AMD is the most common cause of visual impairment leading to irreversible blindness. AMD can develop into two different forms: the neovascular form (i.e. “wet” AMD) or the non-neovascular form (i.e. “dry” AMD or geographic atrophy). Dry AMD is characterized by the presence in the macula of drusen, deposits of extracellular debris that lead to retinal pigment epithelium (RPE) loss and photoreceptor cell death. Wet AMD is characterized by neovascularization of the choriocapillaris from the Bruch's membrane into the subretinal space, which can result in photoreceptor cell death, RPE detachment and blindness. Wet AMD accounts for only 10-20% of AMD cases, but is responsible for the majority of the severe vision losses associated with AMD. There is unmet medical need since no treatments are currently available for dry AMD, while treatments for wet AMD are not satisfactory. Hence, several wet AMD patients are refractory to anti-VEGF treatments, while chronic usage of anti-VEGF increases the risk to develop geographic atrophy (or dry AMD).

Studies have highlighted the role of Vascular Endothelial Growth Factor (VEGF) in pathological choroidal neovascularization. VEGF is a key factor for the activation of angiogenesis by stimulating the migration and proliferation of endothelial cells (ECs) as well as by preventing apoptosis and regulating vascular permeability in endothelial cells. Currently, the prescribed therapy for wet AMD consists of VEGF inhibitors intra-vitreal administration to stop choroidal neovascularization. Treatments aimed at stopping abnormal blood vessel growth include FDA-approved drugs such as Lucentis™, Eylea™ and Macugen™, which block excessive blood vessel growth by inhibiting VEGF signaling. However, treatments involve frequent intraocular injections, and a proportion of patients do not achieve vision improvement. Furthermore, anti-VEGF therapy has not shown the ability to fully eradicate choroidal neovascularization (CNV), so that recurrences are common when the intravitreal injections are suspended. Accordingly, there is a need for a treatment of wet AMD that involves less frequent intraocular injections and increased efficiency than current existing treatment.

The dependence of non-vascular cells on VEGF signaling in the eye has also raised concerns that long-term VEGF inhibition may adversely affect the retina. Recent studies showed expression of VEGF and VEGFR2 in the retina and demonstrated the importance of their signaling for the survival of non-vascular retinal cells such as Müller cells and photoreceptors, not only during development but also during adulthood. As shown by Saint-Geniez and collaborators, VEGF neutralization leads to increased retinal apoptosis in mice, which results in decreased inner and outer retinal nuclear layer thickness. Clinical studies have also shown that long-term inhibition of VEGF signaling in patients was associated with increased risks of geographic atrophy. Therefore, there is a need for treatments that block ocular neovascularization without adversely affecting non-vascular cells will constitute a significant tool to counter pathological angiogenesis in wet AMD. Treatments not acting on VEGF signaling are thus of major interest to be used alone or in combination with reduced concentration of anti-VEGF drugs for the treatment of wet AMD.

DAND5 (also called CER2 and COCO) is a member of the Cerberus family, composed of secreted proteins which act as antagonists of BMP, TGFbeta, and Wnt signaling molecules and are involved in establishing anterior-posterior patterning in vertebrates. Seven Cerberus family genes have been identified: NBL1 (DAN), GREM1 (DAND2), GREM2 (DAND3), CER1 (DAND4), DAND5 (Coco), SOST (DAND6) and SOSTDC1 (DAND7)). These seven proteins are members of the DAN family of BMP antagonists.

Inactivation of DAND5 in mice leads to multiple laterality and cardiovascular defects and a significant proportion of animals die perinatally. A recent study has shown that DAND5 is widely expressed in the retinal photoreceptor layer, and that it is a potent inducer of cone photoreceptor differentiation and maintenance (16). Notably, DAND5 can promote the differentiation of human pluripotent stem cells into cone photoreceptors (16). DAND5 also displays pro-survival activities on stem cell-derived human cones in vitro (unpublished work). However, prior to the present invention, it was unknown that DAND5, or any of the six other members of the DAN family of BMP antagonists, could play a role in inhibiting and preventing ocular neovascularization and pathological angiogenesis.

Accordingly, there is a need for an innovative therapeutic approach for the treatment of ocular conditions, including ocular condition and diseases resulting from ocular neovascularization such as AMD, diabetic retinopathies, retinopathy of prematurity, and other ocular diseases associated with undesirable angiogenesis and/or undesirable vasculogenesis.

There is also a need for therapeutic methods and pharmaceutical compositions that can fully eradicate choroidal neovascularization and achieve vision improvement without modifying the mature vascular networks of the eye, without creating retinal apoptosis and/or without increasing the risks of retinal geographic atrophy in the course of long-term treatments.

There is also a need for an innovative therapeutic approach having trophic effects to promote survival, growth, differentiation, and synaptic plasticity of endogenous and/or grafted photoreceptors of the eye for the treatment of retinal degenerative diseases or lesions affecting photoreceptors.

The present invention addresses these needs and other needs as it will be apparent from reviews of the disclosure and description of the features of the invention hereinafter.

### BRIEF SUMMARY OF THE INVENTION

The present invention is concerned with prevention and/or treatment of visual impairment and vision loss, and more particularly to the use of DAN family BMP antagonist(s) and methods thereof for the prevention of ocular neovascularization and, as a trophic factor for photoreceptors in eye diseases.

According to one aspect, the invention relates to a method for treating an ocular condition, comprising administering to a mammalian subject in need thereof an effective amount of a DAN family BMP antagonist.

According to another aspect, the invention relates to a method for long-term inhibition of neovascularization in an ocular condition, comprising administering to a mammalian subject in need thereof an effective amount a DAN family BMP antagonist.

According to another aspect, the invention relates to a method of replacement therapy for vascular endothelial growth factor (VEGF) inhibitor treatment, the method comprising administering to a mammalian subject in need thereof an effective amount of a DAN family BMP antagonist instead of administering a VEGF inhibitor.

According to another aspect, the invention relates to a method for inhibiting and/or preventing ocular neovascularization and/or ocular angiogenesis in a mammalian subject, the method comprising administering to a mammalian subject in need thereof an effective amount of a DAN family BMP antagonist.

According to another aspect, the invention relates to a pharmaceutical composition for treating an ocular condition, comprising a DAN family BMP antagonist, and a pharmaceutically acceptable carrier or excipient.

According to another aspect, the invention relates to the use of a DAN family BMP antagonist, for the treatment of an ocular condition.

According to another aspect, the invention relates to the use of a DAN family BMP antagonist in the manufacture of a medicament for the treatment of an ocular condition.

According to another aspect, the invention relates to the use a DAN family BMP antagonist in retinal transplantation therapy.

According to another aspect, the invention relates to an improve method of retinal cell transplantation for treating an ocular condition comprising administering retinal cells to a mammalian subject, the improvement comprising at least one of: (i) coating said retinal cells with a DAN family BMP antagonist; (ii) incorporating said retinal cells in a matrix comprising a DAN family BMP antagonist; and (iii) injecting a DAN family BMP antagonist into the vitreous at the time of the retinal cell transplantation and/or shortly thereafter.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

In order that the invention may be readily understood, embodiments of the invention are illustrated by way of example in the accompanying figures.

FIG. 1 is a panel with pictures, bar graphs, and graphs showing that Dand5 reduces VEGF-mediated sprouting angiogenesis in cultured endothelial cells. FIG. 1A: HUVECs grown in a fibrinogen gel were treated with VEGF to induce tube formation in the presence or absence of Dand5. Dand5 inhibited tube formation in a dose dependent manner. FIG. 1B: HUVECs were plated to confluency and scratched to stimulate a wound in the presence or absence of Dand5. Scale bar, 100 μm. FIG. 1C: Quantification of tube formation in FIG. 1A n=3, *p<0.05, **p<0.01). FIG. 1D: Quantification of wound closure shown in FIG. 1B (n=6,***p<0.0001). FIG. 1E: Choroids were isolated from wild-type mice and cultured in vitro in the absence or presence of Dand5. Choroids were stained with calcein AM dye and fluorescent images were acquired (representative image of n=3). FIG. 1F: Quantification of sprouting areas in choroidal sprouts (n=3, ***p<0.0001). FIG. 1G: Neutral red assay examining growth of HUVEC cells (n=3, *p<0.05). FIG. 1H: HUVECs were cultured in the presence or absence of Dand5 and analyzed by immunofluorescence using antibodies against phospho-Histone 3 (PH3), a marker of cellular mitosis, and phalloidin and DAPI (nuclei). White arrows indicate phospho-Histone 3 positive cells. Scale bar, 50 μm. FIG. 1I: Quantification immunofluorescent staining of phospho-histone 3 positive cells. (n=2, ***p<0.0001). FIG. 1J: Immunofluorescent imaging of cleaved caspase 3 (CC3), phalloidin and DAPI (nuclei) in HUVEC cells cultured in the presence or absence of Dand5. White arrows indicate cleaved caspase 3 positive cells. Scale bar, 50 μm. FIG. 1K: Quantification of immunofluorescent staining of cleaved caspase 3 (n=2, n.s.).

FIG. 2 is a panel with schematics, pictures, and bar graphs showing that Dand5 inhibits retinal angiogenesis and increases vessel instability during retinal vascular development. FIG. 2A: Schematic representation of experimental set-up (A-F). Neonatal pups received intravitreal injections of either PBS, Dand5, Flt1-Fc on P1. Retinas were harvested on P5. FIG. 2B: Isolectin B4 staining of flatmount retinas, scale bar, 100 μm. FIG. 2C: Quantification of branch points and vessel length (n=6 mice/group; *p<0.05, **p<0.01). FIG. 2D: Immunofluorescent images of flatmount retinas were stained with antibodies against Collagen IV and isolectin B4. Empty collagen sleeves (white arrows) are increased in the presence of Dand5. FIG. 2E: Immunofluorescent images of pericytes, NG2+ cells, surrounding the retinal vessels (marked with IsoB4). FIG. 2F: Immunostaining of cleaved caspase 3 (green), s-opsin (red) and DAPI (blue) on retinas treated with either PBS or Dand5. White arrows indicate a cleaved caspase 3-positive cell present in the inner nuclear layer. FIG. 2G: Quantification of apoptotic cells (CC3+) in the peripheral and central retina of mice treated with PBS (black bars) or Dand5 (grey bars) (n=4, ns).

FIG. 3 is a panel with a schematic, pictures, and bar graphs showing that acute injection of Dand5 is not detrimental to mature retinal vasculature. FIG. 3A: Schematic representation of experimental set up (B-C). Mice of 6 weeks of age received a single intravitreal injection of PBS, Dand5 (60 ng/mL) and were analyzed 5 days following injection. FIG. 3B: Isolectin B4 staining of flatmount retinas. Scale bar, 100 μm. FIG. 3C: Quantification of vessel length and vessel branching of mice described in FIG. 1F. DAND5 is not detrimental to mature vessels.

FIG. 4 is a panel with a schematic, images and bar graphs showing that chronic intravitreal injections of Dand5 inhibits developmental angiogenesis but is not detrimental to photoreceptor survival. FIG. 4A: Schematic representation of experimental set-up (B-E). Neonatal pups received intravitreal injections of either PBS, Dand5, Flt1-Fc on P1, P7 and P14. Retinas were harvested on P28. FIG. 4B: Isolectin B4 staining of flatmount retinas (P28) described in FIG. 3A. FIG. 4C: Quantification of branch points and vessel length in retinas described in FIG. 3B (n=6 mice/group; *p<0.05, **p<0.01). FIG. 4D: Immunofluorescent images of sectioned retinas described in FIG. 3A stained with antibodies against s-opsin and rhodopsin and DAPI (nuclei). FIG. 4E: Quantification of the number of nuclei present in the outer nuclear layer of retinas treated as described in (4A) (n=4, ns).

FIG. 5 is a panel with a schematic, images and bar graphs showing that Dand5 inhibits pathological vessels growth in a mouse model of choroidal neo-vascularization FIG. 5A: Schematic representation. Pups at P7 were placed under hyperoxic conditions (75% oxygen) until P12. At day P12, mice received a single intravitreal injection of PBS, Dand5 or Flt1-Fc and were housed under conditions of normoxia (normal room air). Retinas were harvested 3 days later on day P17. FIG. 5B: Representative images of flatmount retinas were stained with isolectin B4. Neovascular area is highlighted. FIG. 5C: Quantification of neovascular area of retinas (n=7 (PBS); n=8 (Dand5); n=5 (Flt1Fc). * p<0.05; ** p<0.01.). FIG. 5D: Schematic representation. Adult mice of 6 weeks of age received intravitreal injections of either PBS, Dand5 or Flt1-Fc. Mice were then subjected to laser burn (400 mW intensity, 0.05 s exposure) to disrupt the Bruch's membrane. Eyes were harvested two weeks later. FIG. 5E: Immunofluorescent staining of eyes. The RPE is marked using phalloidin, while the choroidal vessels are indicated by isolectin B4. FIG. 5F: Quantification of neovascular area from eyes (n=7 (control); n=6 (Dand5); n=4 (Flt1 Fc). Four burns per choroid were quantified, * p<0.05.

FIG. 6 is a panel with pictures and graphs showing that Dand5 does not significantly affect VEGF signaling in cultured HUVECs. FIG. 6A: HUVECs were grown in complete media were in the absence or presence of Dand5 or IWR1 for up to 24 hours. Protein extracts were analyzed by immunoblotting (representative image of n=3 experiments). FIG. 6B: Quantification of immunoblots presented in FIG. 6A (n=3, ns). FIG. 6C: HUVECs were grown in low serum media for 16 h in the absence or presence of 60 ng/mL of Dand5. VEGF signaling was induced by addition of 25 ng/mL of rhVEGF for up to 60 minutes. Protein extracts were analyzed by immunoblotting (representative image of n=3). FIG. 6D: Quantification of the mean gray value of phosphorylated proteins normalized to the mean gray value of β-Actin of immunoblots in FIG. 6C (n=3, ns). FIG. 6E: HUVECs were plated in the presence or absence of 60 ng/mL of Dand5. Proteins extracts were analyzed for expression of VEGF receptors (representative image of n=3). FIG. 6F: Quantification of immunoblots presented in FIG. 6E (n=3, ns).

FIG. 7 is a panel with a schematic and graphs showing that DAND5 modifies the metabolic transcriptional program in cultured endothelial cells. HUVECs were treated for 6 and 16 h with either PBS or DAND5, before RNA extraction. RNA was reverse-transcribed and cDNA amplified using IonTorrent Ampliseq™ Transcriptome Human Gene Expression kit. FIG. 7A: Heat map of metabolism-related genes. FIG. 7B: Volcano plot highlighting in red the subset of genes significantly modified following DAND5 treatment. Note down-regulation of LDHA in DAND5-treated HUVECs (7A).

FIG. 8 is a panel with graphs and pictures showing that Dand5 induces mitochondrial oxidative stress and alters ATP levels in HUVECs. FIG. 8A: HUVEC cells were treated with Dand5 in the presence or not of N-acetylcysteine (NAC) for up to 48 hours. Cells were then incubated with H₂DCFDA. Hydrogen peroxide treated cells (H₂O₂) were included as a positive control. Fluorescence was measured at Ex: 485 nm and Em: 535 nm in a plate reader. Relative Fluorescence Units (RFU) were generated by normalizing the reading of each well to Hoescht staining (n=3,*p<0.05 **p<0.001). FIG. 8B: HUVEC cells were treated with Dand5 in for up to 48 hours. Cells were incubated with MitoSox Red. 3-nitro-proprionic acid (3-NP) treated cells were also examined as a positive control. Fluorescence was measured at Ex: 544 nm and Em: 590 nm. Relative Fluorescence Units (RFU) were generated by normalizing the reading of each well to Hoescht staining (n=3,*p<0.05). FIG. 8C: Primary culture of choroidal sprouts were treated with Dand5 in the absence or presence of NAC. FIG. 8D: Quantification of sprouting area by Image J. FIG. 8E: The ratio of NAD+ to NADH in whole cell extracts was determined using an Alcohol Dehydrogenase/Malate Dehydrogenase enzymatic cycling assay. Cells treated to hypoxic conditions (4% O₂ for 3 hours) were included as a positive control (n=3,*p<0.05, **p<0.01). Likewise, glucose uptake was reduced in Dand5-treated HUVECs after 1 hour, while lactate levels were increased (lower panel). FIG. 8F: ATP levels were reduced in HUVECs exposed to Dand5 at 1, 6 and 24 hours (n=3,*p=0.05).

FIG. 9 is a panel with pictures showing that Dand5 co-localizes with mitochondria one hour after treatment in HUVECs. FIG. 9: HUVECs were plated to confluence and exposed or not to Dand5 for 1 hour. Dand5 was not detected before treatment in HUVECs (not shown). After 1 hour of treatment, Dand5 was found to co-localizes with mitochondria, as determined using an antibody against Dand5 (anti-human Dand5 antibody) and an anti-human mitochondria antigen antibody (arrows).

FIG. 10 is a panel with a schematic, pictures and graphs showing that Dand5 does not affect cellular polarity during wound healing. FIG. 10A: HUVECs were plated to confluence and then scratched to induce a wound. Cells were then immuno-stained with GM130 (green) to mark the golgi and phalloidin (red) and DAPI (blue). White arrows indicate the directionality of the Golgi. FIG. 10B: Definition of polarized cells. FIG. 10C: Quantification of polarized and non-polarized cells (n=2, ns). FIG. 10D: HUVECs grown in 1% FBS and the absence or presence of Dand5. Proliferating cells incorporated EdU and were sorted by FACS (n=2, **p<0.01). FIG. 10E: Representative FACS plots of HUVECs sorted by EdU (representative of n=2). FIG. 10F: Annexin V and propidium iodide (PI) FACS analysis in HUVEC treated for 24 hours with Dand5, Ceramide or Cisplatin (representative of n=3). FIG. 10G: FACS plots of HUVECs sorted by PI and Annexin V (representative of n=3).

FIG. 11 is a panel with a schematic, pictures and graphs showing that intravitreal injection of Dand5 reduces cellular mitosis but does not affect apoptosis during retinal vascular development. FIG. 11A: Schematic representing experimental design. FIG. 11B: Representative images of flatmount retinas stained with isolectin B4 (magenta) and phospho-histone 3 (green). White arrows denote pH3+ ECs. FIG. 11C: Quantification of pH3-positive cells normalized to the vascular area (n=11 (PBS), n=3 (Dand5); Student's t-test, ***p<0.005). FIG. 11D: Representative images of flatmount retinas stained with isolectin B4 (magenta) and cleaved caspase 3 (green). Arrowheads denote CC3+ ECs. FIG. 11E: Quantification of CC3+ cells normalized to blood vessel area; n=5 (PBS), n=3 (Dand5); Student's t-test, ns). FIG. 11F: Representative images of flatmount retinas stained with isolectin B4 (magenta) and Rip3 (green). White arrows denote Rip3+ ECs. FIG. 11G: Quantification of Rip3-positive cells in retinas prepared as described in 11A.

FIG. 12 is a panel with a schematic, pictures showing that Dand5 reduces canonical Wnt signaling in HUVECs. HUVECs were plated to confluence and received a 2 h scratch in the presence or absence of Dand5 (60n/mL) or IWR1 (3 μM). β-catenin (green) localization was examined by immunofluorescence with phalloidin and DAPI (nuclei) to identify the cell boundaries and nucleus. White arrows indicate cells with increased cytoplasmic β-catenin in Dand5 and IWR1-treated cells. Scale bars, 10 μM).

FIG. 13 is a panel with pictures and graphs showing that Dand5 opposes Wnt1-mediated hypervascularization during retinal vascular development. FIG. 13A: Schematic representation of experimental design. C57B6 pups were injected at P1 with PBS, Dand5 (60 ng/mL), Wnt1, or Wnt1 and Dand5 together at various molar ratios (1N, 5N and 10N). FIG. 13B: Representative images of retinas treated with PBS, Dand5 (1N) or Wnt1 (1N). FIG. 13C: Representative images of retinas treated with PBS or with Wnt1 (5N and 10N) alone or in combination with Danb5. Dand5 Flatmounts retinas were stained with Isolectin B4 to visualize the vasculature. FIG. 13D: Quantification of vascular branches in treated eyes. Note that Wnt1 and Dand5 display mutually antagonist activities.

FIG. 14 is a panel with schematics showing that Dand5 is a therapeutic molecule for retinal cell transplantation therapy. FIG. 14A: Schematic representation of trans-vitreal injection of human photoreceptors and/or RPE in the sub-retinal space of a patient with retinal and/or macular degeneration. FIG. 14B: Schematic representation of human photoreceptors grafted in the sub-retinal space of a patient having lost its photoreceptors (onl). The graft is surrounded by human recombinant Dand5 molecules that are released by a Hyaluronidase (HA) gel. FIG. 14C: Dand5 is a bio-active, therapeutic molecule, that promotes photoreceptors and RPE cells survival, while inhibiting retinal and choroidal neo-vascularization.

FIGS. 15 to 21 provide the nucleic acid sequence and the amino acid sequence of NBL1 (DAN) (FIG. 15), GREM1 (DAND2) (FIG. 16), GREM2 (DAND3) (FIG. 17), CER1 (DAND4) (FIG. 18), DAND5 (Coco) (FIG. 19), SOST (DAND6) (FIG. 120) and SOSTDC1 (DAND7) (FIG. 21).

Further details of the invention and its advantages will be apparent from the detailed description included below.

### DETAILED DESCRIPTION OF EMBODIMENTS

In the following description of the embodiments, references to the accompanying figures are by way of illustration of an example by which the invention may be practiced. It will be understood that other embodiments may be made without departing from the scope of the invention disclosed.

The invention relates to the medical uses of DAN family BMP antagonists in the treatment of ocular conditions. To date, the DAN family BMP antagonists comprises the seven members identified in Table 1:

TABLE 1
DAN family of BMP antagonists

| Approved | Approved | Previous | | |
|---|---|---|---|---|
| Symbol | Name | Symbols | Synonyms | Chromosome |
| NBL1 | neuroblastoma 1, DAN family | | D1S1733E, DAND1, | 1p36.13 |
| | BMP antagonist | | NO3, DAN, NB | |
| GREM1 | gremlin 1, DAN family BMP | CKTSF1B1, | DRM, HMPS, gremlin, | 15q13.3 |
| | antagonist | CRAC1 | DAND2 | |
| GREM2 | gremlin 2, DAN family BMP | | DAND3, CKTSF1B2, | 1q43 |
| | antagonist | | Prdc, FLJ21195 | |
| CER1 | cerberus 1, DAN family BMP | | DAND4 | 9p22.3 |
| | antagonist | | | |
| DAND5 | DAN domain BMP antagonist | | FLJ38607, CKTSF1B3, | 19p13.13 |
| | family member 5 | | DANTE, GREM3, | |
| | | | CER2, DTE, Coco | |
| SOST | sclerostin | | DAND6, VBCH | 17q21.31 |
| SOSTDC1 | sclerostin domain containing 1 | | USAG1, DAND7, | 7p21.2 |
| | | | DKFZp564D206 | |

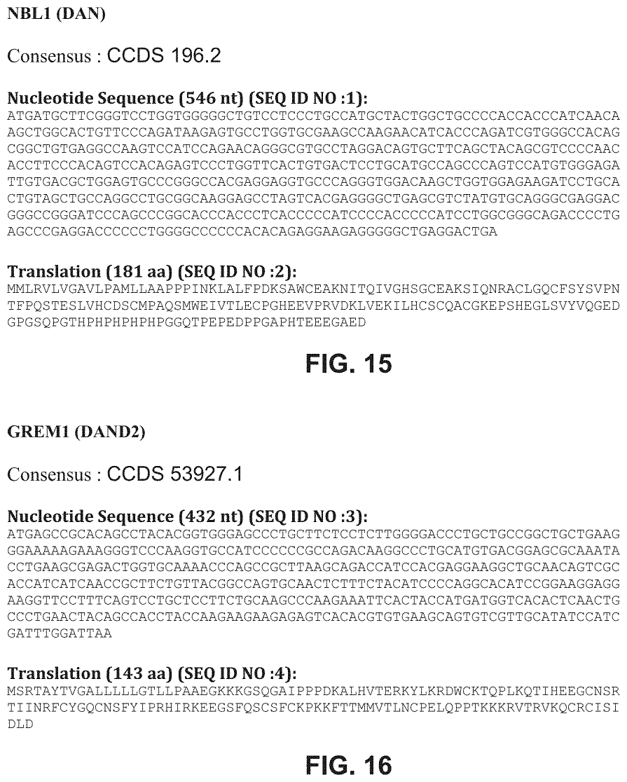

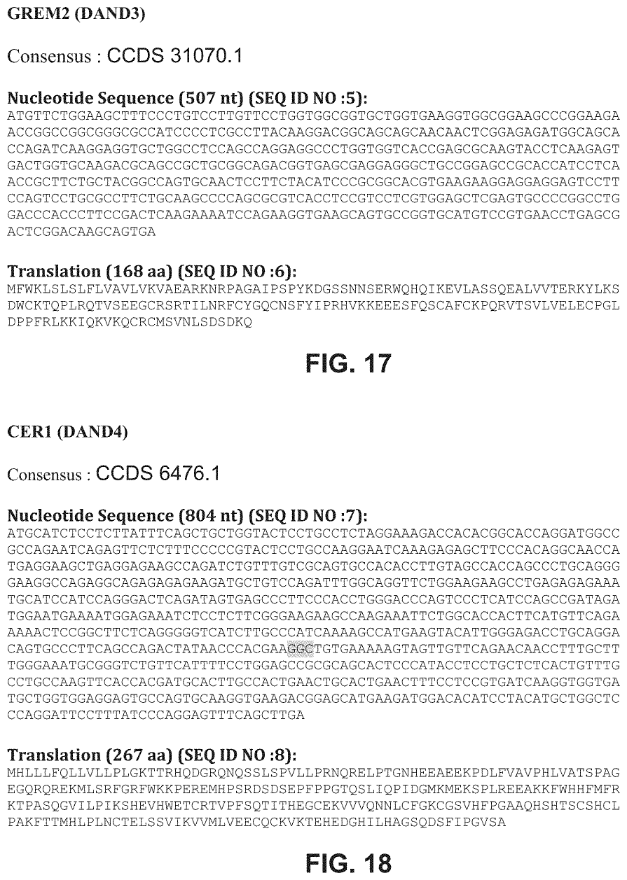

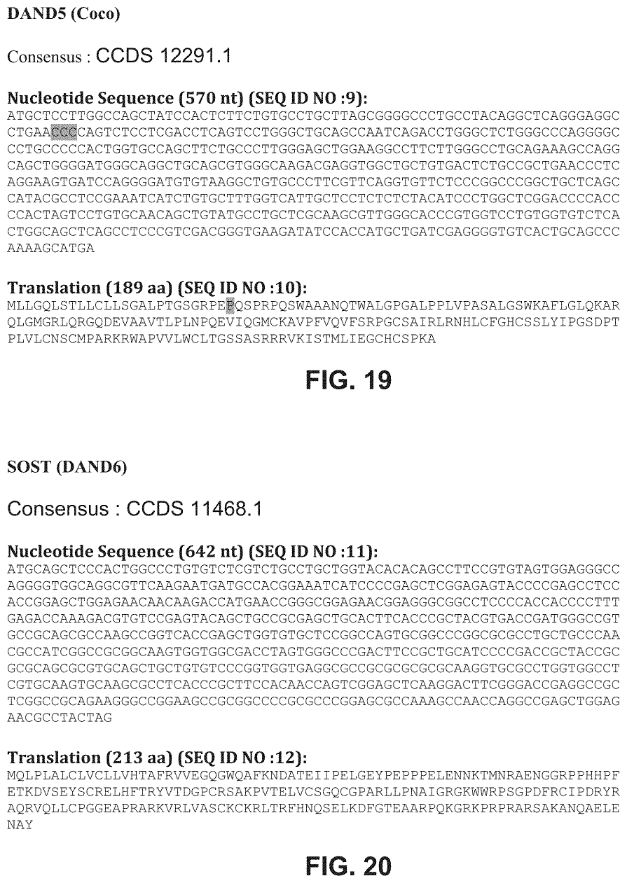

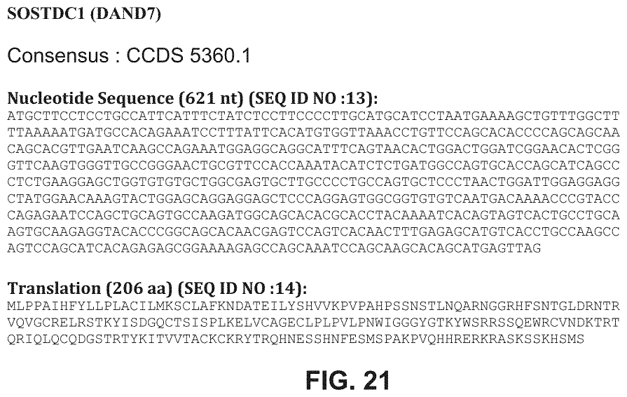

The amino acid sequence of each of the DAN family BMP antagonists is known and publicly available as shown in Table 2 below and provided in FIGS. 15 to 21

TABLE 2
DAN family of BMP antagonists

| Approved | NCBI | Human cDNA | NCBI protein | cDNA | Protein |
|---|---|---|---|---|---|
| Symbol | gene ID | accession number | accession number | SEQ ID NO: | SEQ ID NO: |
| DAND5 | 199699 | CCDS 12291.1 | NP_689867.1 | 9 | 10 |
| | | NM_152654.2 | | | |
| CER1 | 9350 | CCDS 6476.1 | NP_005445.1 | 7 | 8 |
| (DAND4) | | NM_005454.3 | | | |
| NBL1 | 4681 | CCDS 196.2 | NP_001265095.1 | 1 | 2 |
| (DAN) | | NM_001278166.1 | | | |
| GREM1 | 26585 | CCDS53927.1 | NP_001178252.1 | 3 | 4 |
| (DAND2) | | NM_001191323.2 | | | |
| GREM2 | 64388 | CCDS 31070.1 | NP_071914.3 | 5 | 6 |
| (DAND3) | | NM_022469.4 | | | |
| SOST | 50964 | CCDS 11468.1 | NP_079513.1 | 11 | 12 |
| (DAND6) | | NM_025237.3 | | | |
| SOSTDC1 | 25928 | CCDS 5360.1 | NP_056279.1 | 13 | 14 |
| (DAND7) | | NM_015464.3 | | | |

When used in connection with the treatment of ocular conditions (and related aspect of the present invention such as inhibition of neovascularization, inhibiting angiogenesis, etc.), the term “DAN family BMP antagonist” refers to a full-length protein selected from NBL1 (DAN), GREM1 (DAND2), GREM2 (DAND3), CER1 (DAND4), DAND5 (Coco), SOST (DAND6) and SOSTDC1 (DAND7). The term also encompasses biologically active variants of the full-length proteins and biologically active fragments thereof having BMP antagonist activity similar (preferably at least equivalent or superior) to the full-length proteins.

A biologically active variant of a full-length DAN family BMP antagonist in accordance with the present invention may comprise an insertion, a deletion or an amino acid substitution (conservative or non-conservative) with respect to the reference full-length DAN family BMP antagonist set forth in SEQ NOs: 1 to 7.

A biologically active variant of a full-length DAN family BMP antagonist in accordance with the present invention may comprise at least 80%, 81%, 82%, 83%, 84%, 85%, 86%, 87%, 88%, 89%, 90%, 91%, 92%, 93%, 94%, 95%, 96%, 97%, 98%, 99% or more sequence identity with the sequence of the full-length protein or a portion thereof, said portion comprising at least 50, 75, 100, 125, 150, 175, 200, 225, 250, 275, 300, 325 or more amino acids.

A biologically active variant of a full-length DAN family BMP antagonist in accordance with the present invention may comprise at least 80%, 81%, 82%, 83%, 84%, 85%, 86%, 87%, 88%, 89%, 90%, 91%, 92%, 93%, 94%, 95%, 96%, 97%, 98%, 99% or more sequence similarity with the sequence of the full-length protein or a portion thereof, said portion comprising at least 50, 75, 100, 125, 150, 175, 200, 225, 250, 275, 300, 325 or more amino acids.

A biologically active fragment of a full-length DAN family BMP antagonist in accordance with the present invention may comprise at least 50, 75, 100, 125, 150, 175, 200, 225, 250, 275, 300, 325 or more contiguous amino acids of any one of SEQ ID NOs: 1 to 7.

Preferred biologically active variants and fragments preferably comprise the same or at least similar biological activity than other members of CAN (Cerberus and Dan) subfamily of BMP antagonists, to which DAND5 belongs, including and at least one desirable biological activity including but not limited to, the biological activities listed hereinafter. For instance, biologically active variants and fragments in accordance with the present invention may comprise C-terminal cystine knot with an eight-membered ring, and/or form homo- and heterodimers. Biologically active variants and fragments may possess the same or similar antagonistic effects than the original secreted protein, including, but not limited to, antagonistic effects associated with direct binding to BMP proteins. Biologically active variants and fragments may play a role in regulating organogenesis, body patterning, and tissue differentiation. Biologically active variants and fragments may also bind Nodal and to inhibit the Nodal signaling pathway which patterns left/right body asymmetry.

In embodiments, the full-length protein, variant, or fragment of the DAN family BMP antagonist comprises at least one, preferably two, three or more, of the following biological activities:
i) suppress VEGF-induced tube formation of human umbilical vein endothelial cells (HUVECs);
ii) prevents VEGF-induced cell migration of HUVECs;
iii) inhibits sprouting, migration and/or cellular proliferation of HUVECs;
iv) inhibits retinal blood vessel development;
v) reduction of HUVECs proliferation without causing apoptosis or necroptosis;
vi) inhibits retinal neovascularization;
vii) reduces pathological neovascularization in retina;
viii) prevents choroidal neovascularization (CNV) in a mice model of laser-induced CNV;
ix) inhibits Wnt signaling in HUVECs;
x) inhibits Wnt neo-vascular effect in the mouse retina;
xi) induces elevation of free radicals in HUVECs;
xii) affects mitochondrial oxidative metabolism to decrease the NAD+/NADH ratio and ATP production;
xiii) blocks Glucose uptake and increases Lactate production in HUVECs;
xiv) localizes to mitochondria in treated HUVECs; and
xv) promote differentiation and survival of human photoreceptors and retinal pigment epithelium (RPE).

It is within the skill of those in the art to determine whether a full-length protein, variant, or fragment of the DAN family BMP antagonist according to the invention possesses one or more of those biological activities in vitro, in vivo, in situ, etc. The exemplification section hereinafter provides numerous methods and assays carried out in vitro and in vivo in which such activities have been or could have been evaluated.

### Methods of Uses

As indicated hereinbefore and exemplified hereinafter, a DAN family BMP antagonist according to the invention has beneficial therapeutic and pharmaceutical properties. Therefore, DAN family BMP antagonists may have useful pharmaceutical applications in the treatment of various ocular conditions in mammalian subjects.

A DAN family BMP antagonist according to the invention may also be used in methods of replacement therapy for replacing the use of vascular endothelial growth factor (VEGF) inhibitor(s).

A DAN family BMP antagonist according to the invention may also have useful pharmaceutical applications in long-term inhibition of neovascularization in mammal subject having an ocular condition.

A DAN family BMP antagonist according to the invention may also be useful for inhibiting and/or preventing pathological ocular angiogenesis in a mammalian subject.

As used herein, “neovascularization” refers to the natural formation of new blood vessels usually in the form of functional microvascular networks, capable of perfusion by red blood cells, that form to serve as collateral circulation in response to local poor perfusion or ischemia (e.g. in a pathological context). Neovascularization is conventionally distinguished from “angiogenesis” in that angiogenesis is mainly characterized by the protrusion and outgrowth of capillary buds and sprouts from pre-existing blood vessels.

A DAN family BMP antagonist according to the invention may also be useful for replacing vascular endothelial growth factor (VEGF) inhibitor(s) that are currently being used in the treatment of ocular condition(s). Alternatively, DAN family BMP antagonists may be used in combination with VEGF inhibitor(s) as to increase the anti-neovascular effect or with reduced concentration of VEGF inhibitor(s) as to reduce anti-VEGF adverse effects on retinal cells and RPE. Examples of currently used VEGF inhibitors include, but are not limited to, ranibizumab (Lucentis™), bevacizumab. (Avastin™), Aflibercept (Eylea™), Pegaptanib (Macugen™).

According to preferred embodiments, the DAN family BMP antagonist is used for treating an ocular condition in a mammalian subject in need thereof. The term “mammalian subject” includes mammals in which treatment of an ocular condition is desirable. The term “subject” includes domestic animals (e.g. cats, dogs, horses, pigs, cows, goats, and sheep), rodents (e.g. mice or rats), rabbits, squirrels, bears, primates (e.g., chimpanzees, monkeys, gorillas, and humans), wild animals such as those living in zoos (e.g. lion, tiger, elephant, and the like), and transgenic species thereof. Preferably, the mammalian subject is a human, more preferably a human patient in need of treatment. Even more preferably the mammalian subject is a human patient diagnosed or susceptible to suffer from wet age-related macular degeneration (AMD), retinopathy of prematurity and diabetic retinopathy.

Ocular conditions encompassed by the present invention include, but are not limited to, wet age-related macular degeneration (AMD), dry AMD, retinopathy of prematurity, diabetic retinopathy, ocular neovascularization, Stargardt's disease, inherited retinal dystrophy, cone and cone/rod dystrophy, retinitis pigmentosa, ocular angiogenesis, photoreceptors damage, retinal pigment epithelial cells (RPEC) detachment, and other retinal degenerative diseases affecting photoreceptors. In preferred embodiments, the ocular condition is selected from wet age-related macular degeneration (AMD), retinopathy of prematurity and diabetic retinopathy.

As used herein, the terms “treatment” or “treating” of a subject include administration of DAN family BMP antagonist of the invention, or pharmaceutical composition comprising same, to a subject with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

In one embodiment, the method comprises administering to the subject a therapeutically effective amount of a DAN family BMP antagonist (e.g. full-length protein, variant and/or fragment thereof) as defined herein. In preferred embodiments, the DAN family BMP antagonist is contacted with retinal cells (e.g. photoreceptors, amacrine neurons, bipolar neurons, horizontal neurons, ganglion cells, muller glia, astrocytes and/or RPE) and endothelial cells (pericytes and endothelial vascular cells).

Any suitable method of administration may be used to contact the retinal cells with the DAN family BMP antagonist. In embodiments, the DAN family BMP antagonist is administered using at intravitreal injection and/or subretinal injection Also, transplanted cells could be “coated” or comprised in a matrix and/or in micro-beads comprising the DAN family BMP antagonist, as described hereinafter. Alternative mode of administration of a DAN family BMP antagonist may comprise applying a cream or drops to the cornea of the subject, provided that full-length protein, variant and/or fragment thereof can reach the retinal cells for effecting its desired biological effect.

It may also be possible to obtain beneficial therapeutic effects using genetic and/or recombinant techniques. For instance, it may be possible to deliver the DAN family BMP antagonist to the ocular cells by administering an isolated or purified nucleic acid molecules encoding a DAN family BMP antagonist, and/or by administering a vector or genetically modified cells encoding a DAN family BMP antagonist. For instance, the DAN family BMP antagonist could be provided to the desired ocular location through the use of recombinant or other vehicles to deliver a DNA sequence capable of expression of the DAN family BMP antagonist (e.g. full-length protein, variant or fragment) to the ocular cells of the subject. Vectors, such as viral vectors have been used in the prior art to introduce genes into a wide variety of different target cells. Typically, the vectors are exposed to the target cells so that transformation can take place in a sufficient proportion of the cells to provide a useful therapeutic effect from the expression of the desired polypeptide. The transfected nucleic acid may be permanently incorporated into the genome of each of the targeted cells, providing long-lasting effect, or alternatively the treatment may have to be repeated periodically. A variety of vectors for gene therapy, both viral vectors (AAV and lentivirus) and plasmid vectors, are known in the art.

### Retinal Cell Transplantation

As indicated hereinbefore, the present invention further envisions the use of DAN family BMP antagonist(s) in retinal cell transplantation therapies. Indeed, therapeutic approaches as described herein may be helpful in preventing neo-vascularization and cell death of the graft (and host retinal cells) through the anti-neovascular and pro-neurotrophic activity of the DAN family BMP antagonist.

In embodiments, the present invention is used for the treatment of retinal transplantation of grafted retinal cells and/or retinal transplantation of host retinal cells including, but not limited to, cones, rods, retinal pigment epithelium and/or any other related retinal cells from a donor and/or obtained from human somatic or from pluripotent stem cells.

In one embodiment the DAN family BMP antagonist (e.g. human recombinant DAND5) is released from a gel and/or from beads surrounding a retinal graft (or grafted cells) to allow short-term and medium-term delivery at the site of the graft. In alternative, the retinal cells to be transplanted could be coated with the DAN family BMP antagonist.

In one embodiment the cells to be transplanted are comprised or incorporated in a matrix comprising the DAN family BMP antagonist, such as a gel (e.g. hyaluronidase gel or equivalent) and/or into micro-beads. This may allow to control the release of the active antagonist(s) over a short-term and/o medium-term time period.

In alternative or in complementation, the DAN family BMP antagonist could be injected into the vitreous at the time of the transplantation surgery or shortly thereafter to increase the therapeutic effects of the retinal cell transplantation.

In accordance with the present invention, the DAN family BMP antagonist may be administered alone or in combination with any molecule or compound promoting grafted cells and/or host cells survival in retinal transplantation therapy including, but not limited to Mesencephalic astrocyte-derived neurotrophic factor (MANF), Ciliary neurotrophic factor (CNTF), Brain-derived neurotrophic factor (BDNF), Nerve growth factor (NGF), and Neurotrophin-3 (NT-3).

The present invention encompasses any similar method or technology for releasing in the eye a DAN family BMP antagonist before, after and/or during retinal transplantation therapy.

### Pharmaceutical Compositions

Related aspects of the invention concern pharmaceutical compositions comprising an effective amount a DAN family BMP antagonist as described herein. As used herein, the term “pharmaceutical composition” refers to the presence of at least one DAN family BMP antagonist and at least one pharmaceutically acceptable carrier, diluent, vehicle or excipient.

One particular aspect concerns the use of a therapeutically effective amount of a DAN family BMP antagonist for the treatment of ocular condition(s) in mammalian subjects. Another particular aspect concerns the use of a DAN family BMP antagonist for the long-term inhibition of neovascularization in mammal subjects having an ocular condition. Another particular aspect concerns the use of a DAN family BMP antagonist in the manufacture of a medicament for the treatment an ocular condition. As used herein, the term “therapeutically effective amount” or “effective amount” means the amount of compound that, when administered to a subject for treating or preventing a particular disorder, disease or condition, is sufficient to effect such treatment or prevention of that disorder, disease or condition. Dosages and therapeutically effective amounts may vary, for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g. bioavailability, stability, potency, toxicity, etc.), and the particular disorder(s), disease(s) or condition(s) the subject is suffering from. In addition, the therapeutically effective amount may depend on the severity of the disease state, or underlying disease or complications. Such appropriate doses may be determined using any available assays. When one or more of the DAN family BMP antagonist of the invention is to be administered to humans, a physician may for example, prescribe a relatively low dose at first, subsequently increasing the dose until an appropriate response is obtained. In embodiments, the effective amount for a human subject may comprise a dose of about 0.01 mg to about 2 mg in a single injection volume of about 0.05 ml. In embodiments, the DAN family BMP antagonist of the invention is to be administered for at least 2 weeks, or at least 4 weeks, or at least 6 weeks or at least 8 weeks, or at least 1 year, or at least 3 years, or at least 5 years, or at least 10 years. In one particular embodiment, the DAN family BMP antagonist is administered every 6 weeks, or every 8 weeks, or every 12 weeks, or every 16 weeks, or every 20 weeks. May be used in combination with regular anti-VEGF doses or with anti-VEGF doses 2-3 lower than normal as to reduce anti-VEGF side effects and increase treatment efficacy.

“Pharmaceutically acceptable vehicle” refers to a diluent, adjuvant, excipient, or carrier with which a compound is administered. The term “pharmaceutically acceptable” refer to drugs, medicaments, inert ingredients, etc., which are suitable for use in treatment an ocular condition in mammals (preferably humans) without undue toxicity, incompatibility, instability, irritation, allergic response, and the like, commensurate with a reasonable benefit/risk ratio. It preferably refers to a compound or composition that is approved or approvable by a regulatory agency of the Federal or state government or listed in the U.S. Pharmacopoeia or other generally recognized pharmacopoeia for use in animals and more particularly in humans. The pharmaceutically acceptable vehicle can be a solvent or dispersion medium containing, for example, water, ethanol, polyol (for example, glycerol, propylene glycol, and liquid polyethylene glycol and the like), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms in the composition can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate or gelatin.

The active compound, i.e. DAN family BMP antagonist of the invention, may be formulated prior to administration into pharmaceutical compositions using available techniques and procedures. Formulations of the active compound may be prepared so as to provide a pharmaceutical composition in a form suitable for intravitreal injection subretinal injection, or alternatively, in the form of ointments, creams, drops or the like for ocular administration (see above). The formulation may, where appropriate, be conveniently presented in discrete dosage units and may be prepared by any of the methods well-known in the art of pharmaceutical formulation. All methods include the step of bringing together the active pharmaceutical ingredient(s) with liquid carriers or finely divided solid carriers or both as the need dictates. When appropriate, the above-described formulations may be adapted so as to provide sustained release of the active pharmaceutical ingredient. Sustained release formulations well-known to the art include the use of a bolus injection, continuous infusion, biocompatible polymers or liposomes. In preferred embodiments, the compositions according to the invention are formulated for intravitreal injection and/or subretinal injection.

The method of treatments and compositions of the present invention may also be used in combination with already approved therapies, such as vascular endothelial growth factor (VEGF) inhibitors. Examples of currently approved VEGF inhibitors include, but are not limited to, ranibizumab (Lucentis™), bevacizumab. (Avastin™), Aflibercept (Eylea™), and Pegaptanib (Macugen™).

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following example, which should not be construed as further or specifically limiting.

### EXAMPLES

### Example 1: The BMP Antagonist DAND5/COCO Inhibits Developmental and Pathological Ocular Angiogenesis

The present inventors have hypothesized that DAND5 could play an important role in retinal vascular development by quenching the activity of several members of the BMP, TGFbeta and Wnt families. Findings from the inventors reveal that DAND5 inhibits ocular angiogenesis by a mechanism largely independent of VEGF signaling and through direct activity on endothelial cells mitochondrial metabolism. DAND5 also antagonizes WNT1 activity on retinal vessels when both are co-injected into the mouse eye.

This study evaluates the effects of DAND5 on retinal angiogenesis in developmental and pathological conditions.

Material and Methods

Mice

Adult (3-month old) C57BL/6J (Jax Mice) and P1 to P17 pups mice were used in this study. All animals were housed and bred in a normal experimental room and exposed to a 12-hour light/dark cycle with free access to food and water. All procedures were conducted under the regulation of Canadian federal and institutional guidelines.

Intravitreal Injections

Animals were anaesthetized with isofluorane. A 10 μL Hamilton syringe with a glass-pulled capillary was inserted with a 45° injection angle into the vitreous. When accessing the role of DAND5 developmentally, animals were injected at P1 (DAND5:100 ng; or PBS: 1 μl) and were euthanized at P5 to quantify vascular growth. The DAND5 used for the injection was a human recombinant DAND5/COCO biologically active fragment produced in E. coli and purified by SDS-PAGE (Arg23-Ala189 with an N-terminal Met, predicted 18.2 kDa monomer, R&D systems; 3047-CC-050). During the neovascularization phase of OIR, animals were injected at P12 (2 μL) before sacrifice at P17 for quantification of neovascularization. For adult mice and laser-induced neovascularization, intravitreal injections were performed under a surgical microscope. Mice were anaesthetized with isofluorane. Pupils were dilated using 1% tropicamide. A 33-gauge needle was inserted from the limbus with a 45° injection angle into the vitreous. The direction and location of the needle was monitored through the surgical microscope.

Oxygen-Induced Retinopathy

C57BL/6J mouse pups at postnatal day (P)7 and their fostering mothers (CD1, Charles River) were submitted to 75% oxygen in oxycycler chamber for 5 days. Pups were then returned to normoxia at P12 and administered 100 ng of recombinant human DAND5 intravitreally or similar volume of vehicle in the contralateral eye. Eyes were enucleated at P17 and processed for immunostaining.

Laser-Induced Choroid Neovascularization

Three-month-old C57BL/6J mice were anesthetised with a ketamine/xylazine mix prior to applying a photocoagulating laser (400 mW intensity, 0.05 s exposure time). Four spots were burned around the optical nerve. Mice received 100 ng of recombinant human DAND5 intravitreally or similar volume of vehicle in the contralateral eye. Eyes were enucleated after 14 days and processed for immunostaining.

Immunohistochemistry

Ocular globes were initially fixed for 15 min in 4% paraformaldehyde (PFA). Retinas or choroids were collected after eyes dissection in PBS and blocked 1 h in PBS 3% BSA 0.1% Triton X-100™. Fixation was prolonged in 1% PFA overnight for choroid extraction or eyes sectioning. Prior to sectioning, eyes were maintained in sucrose gradients (10-30%), cryo-preserved in a matrix gel and sliced in 14 μm sections on a cryostat (Leica™ CM3050S). Staining with either FITC-labeled isolectin GS 1B4 (Life technologies corporation), rhodamine phalloidin (Cedarlane Laboratories), phospho-histone H3 (Abcam), Collagen IV (Abcam) or cleaved caspase-3 (Cell Signaling) antibodies were performed on whole and/or sectioned retinas/choroids. Retinas and choroids were then mounted in fluoromount aqueous medium (Sigma-Aldrich). Quantitative analysis of tufts, vaso-obliterated or vessel areas were performed using imageJ/Swift_NV™ as previously described (1). Neovascular tuft formation was quantified by comparing the number of pixels in the affected areas with the total number of pixels in the retina. The avascular area in the retina was measured in the same way using an anti-DAND5/COCO polyclonal goat IgG antibody (AF3047, R&D systems).

Sprouting Assays

Sprouting assays were performed as previously described (2). Briefly, HUVECs (250,000 cells/well in 6-well plates) were resuspended in 300 μl fibrinogen solution (2.5 mg/ml fibrinogen, Sigma-Aldrich) in EBM-2 (Lonza) supplemented with 2% FBS and 50 μg/ml aprotinin (Sigma-Aldrich), and plated on top of a pre-coated fibrin layer (400 μl fibrinogen solution clotted with 1 U thrombin (Sigma-Aldrich) for 20 min at 37° C.). The second layer of fibrin was clotted for 1 hr at 37° C. NHDF cells (250,000 cells/well), in EBM-2 supplemented with 2% FBS and 25 ng/ml VEGF, with or without DAND5 (0, 15, 30 or 60 ng/ml), were then plated on top of the fibrin layers. Cultures were incubated at 37° C., 5% CO₂.

Scratch Assays

Confluent HUVEC monolayers were grown in 6-well plates. Cells were starved 18 hours in EBM-2 medium with 1% FBS. A horizontal wound was created using a sterile 200 μl pipette tip. Next, the cells were washed with EBM2 at 37° C. and incubated in EBM-2 supplemented with VEGF-A (25 ng/ml) or DAND5 (60 ng/ml) at 37° C. for 16 hours. Pictures of scratch wounds were taken just before stimulation (time 0) and after 16 h. Migration % was calculated using ImageJ™ software.

Flow Cytometry

Sub-confluent HUVECs were cultured overnight in starvation medium (EBM2-1% FBS) in the presence or absence of 60 ng/ml DAND5, followed by VEGF stimulation for 1 hour. HUVECs were subjected with a pulse of 5-ethynyl-2′-deoxyuridine (EdU) for 1 hour, and flow cytometry analysis of EdU incorporation was performed as previously described.

Western Blotting

Cells were washed with cold PBS and extracted in Laemmli's buffer, followed by sonication. Samples were run on SDS-PAGE gels and transferred onto nitrocellulose membranes. Membranes were blocked with 5% Bovine Serum Albumin (BSA) and probed with primary antibodies overnight at 4° C.: beta-Catenin (Cell Signaling Technology), phospho-beta-catenin (Cell Signaling Technology), Axin2 (Cell Signaling Technology), beta-Actin (Santa Cruz Technologies). HRP-conjugated secondary antibodies (Vector Laboratories) were used to detect primary antibodies.

NAD+/NADH Enzymatic Cycling and Metabolic Assays

HUVECs plated into 100 mm dishes were treated with COCO for various time points. NAD+ and NADH were extracted using ice cold alkali (0.5M NaOH, 1 mM EDTA) and acidic buffers (0.1M HCl) respectively. Extracts were heated at 60° C. for 30 min and buffers were neutralized with either the NADH (100 mM Tris-HCl pH 8.1, 0.05M HCl) or NAD+(0.4M Tris) neutralization buffers. To attain measurable quantities of NAD+ or NADH, an amplifying cycling assay was performed. Extracts and NAD+ standards were incubated with cycling reagent (67 mM Tris-HCl pH 8, 200 mM EtOH, 1.3 mM beta-mercaptoethanol, 0.01% BSA, 2 mM oxaloacetic acid, 0.5 μg/mL malate dehydrogenase, 5 μg/mL alcohol dehydrogenase) for 1 hour at room temperature. All samples were heated for 5 min at 100 C to stop enzymatic reactions then cooled on ice. For detection, extracts were incubated in an indicator buffer (50 mM 2-amino-2-methyl-propanol pH 9.9, 200 μM NAD+, 10 mM glutamate, 0.04% BSA, 5 μg/mL malate dehydrogenase, 2 μg/mL glutamate oxaloacetate transaminase) for 10 min at room temperature. Then 100 μL if sample was transferred to a black 96-well plate. Fluorescence was detected using a plate reader (TECAN) at an excitation of 365 nm and emission of 460 nm. Standard curves were generated and the concentration of NAD+ and NADH were calculated from the standard curve (references: Kato T., et al. Analytical Biochemistry 53, 86-97 (1973); Lin S., et al, JBC. Vol 276, No. 38, 36000-36007 (2001)).

ATP detection: HUVECs were plated into 100 mm dishes and treated with COCO for various time points. ATP was measured by luminescence (ATP Detection Assay Kit; Cayman Chemicals). Metabolite measurements: HUVECs were cultured for up to 48 hours in the presence or absence of COCO. Culture media was collected at 1 h, 3 h, 6 h, 12 h, 24 h and 48 h, centrifuged at 12000 rpm for 5 min, aliquoted and stored at −80 C. Media was analyzed for glucose concentration using a BioProfiler 400 Analyzer (BioNova). For measurements of lactate, the culture media was analyzed using the Lactate Colorimetric/Fluorometric Assay Kit (BioVision, K607) as per the manufacturer's instructions.

Statistical Analyses

All data are shown as mean±standard error of the mean (SEM). Statistical analyses were performed for all quantitative data using Prism 6.0™ (Graph Pad). Statistical significance for paired samples and for multiple comparisons was determined by Student's t test and ANOVA, respectively. Data were considered statistically significant if the p-value was less than 0.05.

Results

DAND5 Inhibits VEGF-Induced Angiogenesis by Blocking Endothelial Cell Proliferation and Migration

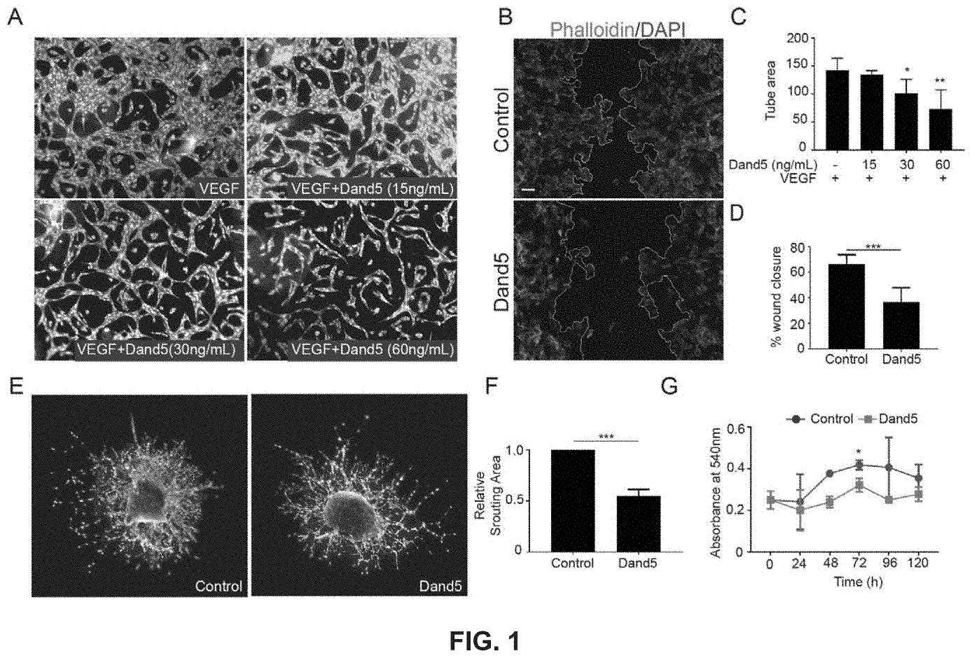

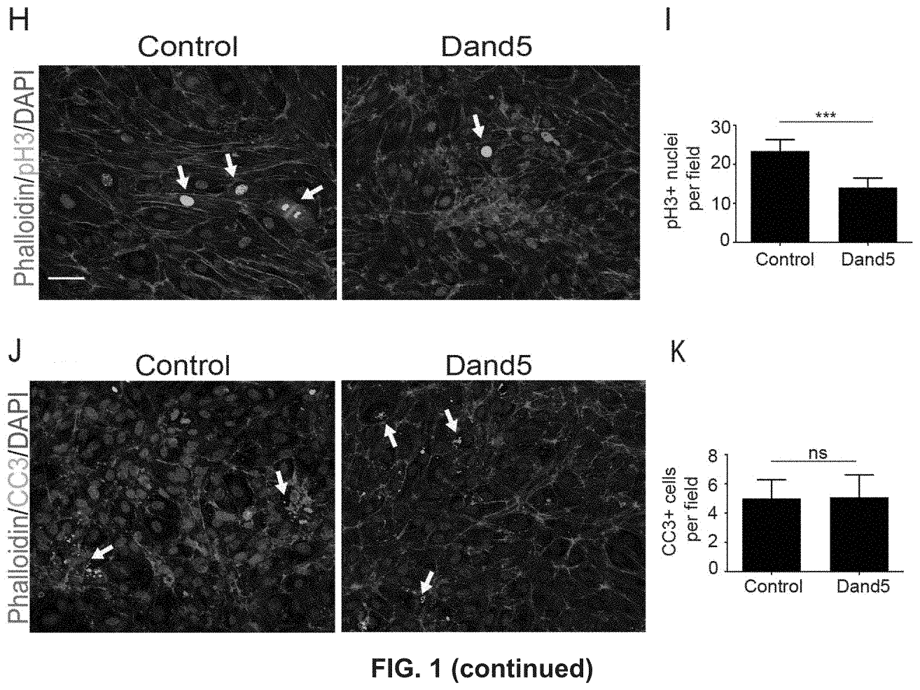

To test whether DAND5 affects sprouting angiogenesis, human umbilical vein endothelial cells (HUVECs) were cultured in 3D fibrin gels and tube formation was induced with VEGF as previously described (2). Recombinant DAND5 protein efficiently suppressed VEGF-induced tube formation in a dose-dependent manner (FIG. 1A). Quantification of endothelial tubes revealed a significant reduction in vascular tube area with increasing concentrations of DAND5 (FIG. 1C).

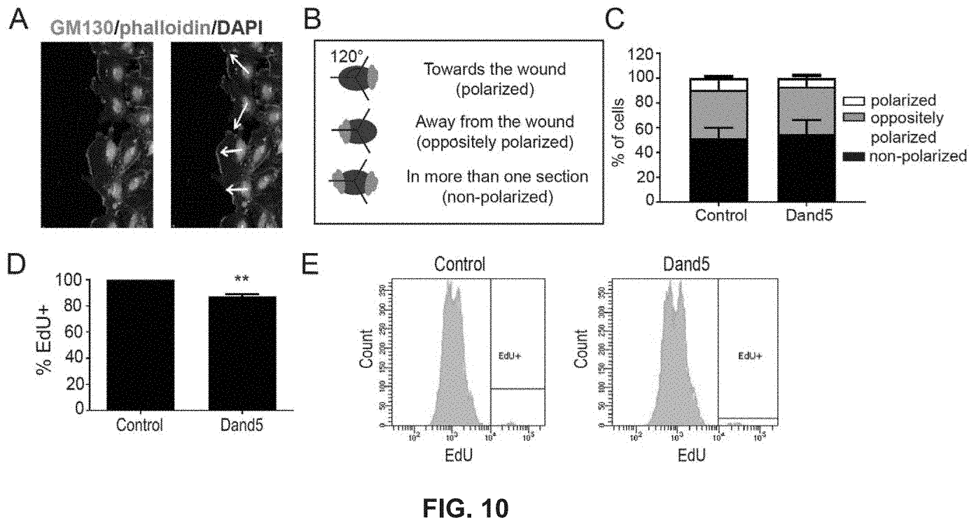

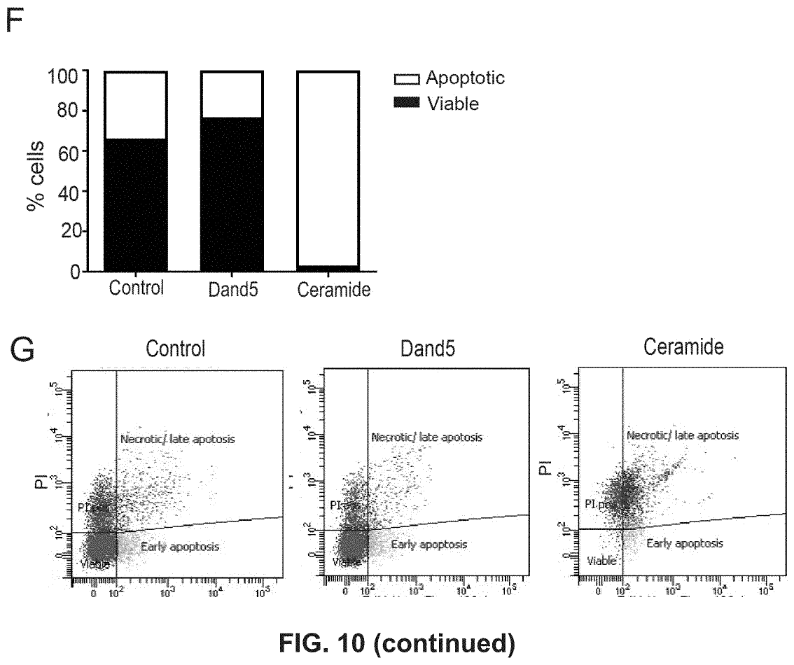

To evaluate the cellular mechanisms underlying the inhibitory effects of DAND5 on endothelial cells sprouting, we assessed the effects of DAND5 on endothelial cell migration and proliferation. Endothelial cell migration plays an essential role in neovascularization, as endothelial tip cells will need to migrate in response to VEGF, and constitutes one of the first steps of the angiogenic response. To address the effects of DAND5 on EC migration, HUVECs were subjected to a wound healing assay. Briefly, a scratch was performed on a confluent monolayer of HUVECs, and wound closure was evaluated at the time of the scratch and again after 18 hours. DAND5 significantly delayed wound closure compared to control treatment, indicating that DAND5 can prevent VEGF-induced cell migration (FIG. 1B, D). Imaging of cells at the wound edge showed that DAND5 did not significantly affect polarization of the Golgi apparatus towards the leading edge, suggesting that DAND5 may not act through Rho GTPase Cdc42 and Rac, which are active at the leading edge of polarized cells and are central to polarity regulation (FIG. 10A-C). DAND5 also inhibited the growth of P5 mouse choroidal explants exposed to serum and VEGF (FIG. 1E, F).

The effects of DAND5 on EC proliferation were assessed by treating sub-confluent HUVECs with complete EC medium for 18 hours with DAND5. Cell mitosis was measured by evaluating the proportion phospho-histone H3 (PH3)-positive ECs following DAND5 treatment. A significant decrease in PH3 staining was observed in DAND5-treated cultures, indicating a lower mitotic index (FIG. 1H, 1). Furthermore, DAND5 prevented the proliferation of cultured ECs, which was demonstrated by culturing HUVECs in the presence or absence of DAND5 and evaluating cell numbers for 6 days by neutral red incorporation (FIG. 1G). Furthermore, Edu-labeling experiments demonstrated that HUVECs cultured in the presence of DAND5 showed reduced Edu incorporation in response to VEGF stimulation (FIG. 10D, E) demonstrating that DAND5 is able of inhibiting endothelial proliferation.

The inhibitory effects of DAND5 on angiogenesis were not associated with increased apoptosis, as HUVECs cultured for 18 hours in the presence of DAND5 did not show differences in the proportion of cleaved caspase 3 (CC3)-positive cells (FIG. 1J, K). Taken together, these data reveal that DAND5 displays anti-angiogenic activity by inhibiting endothelial sprouting, migration and proliferation in vitro.

DAND5 Inhibits Retinal Neovascularization

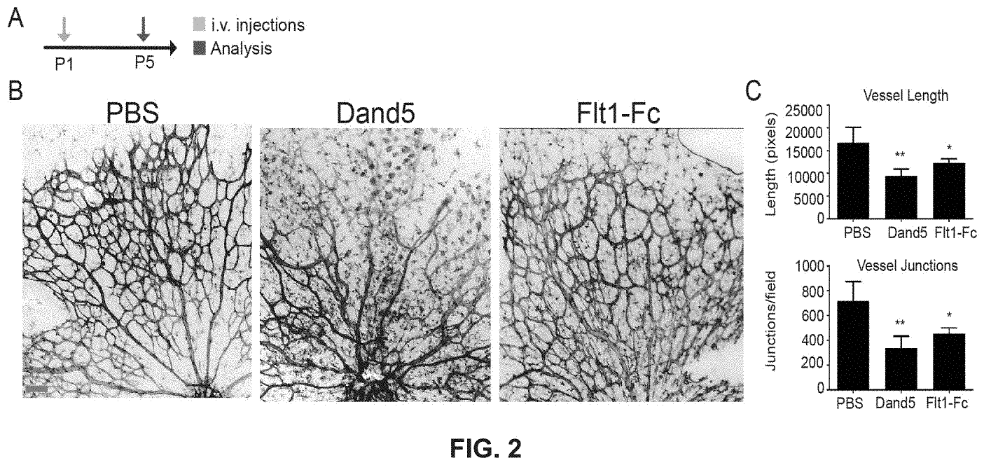

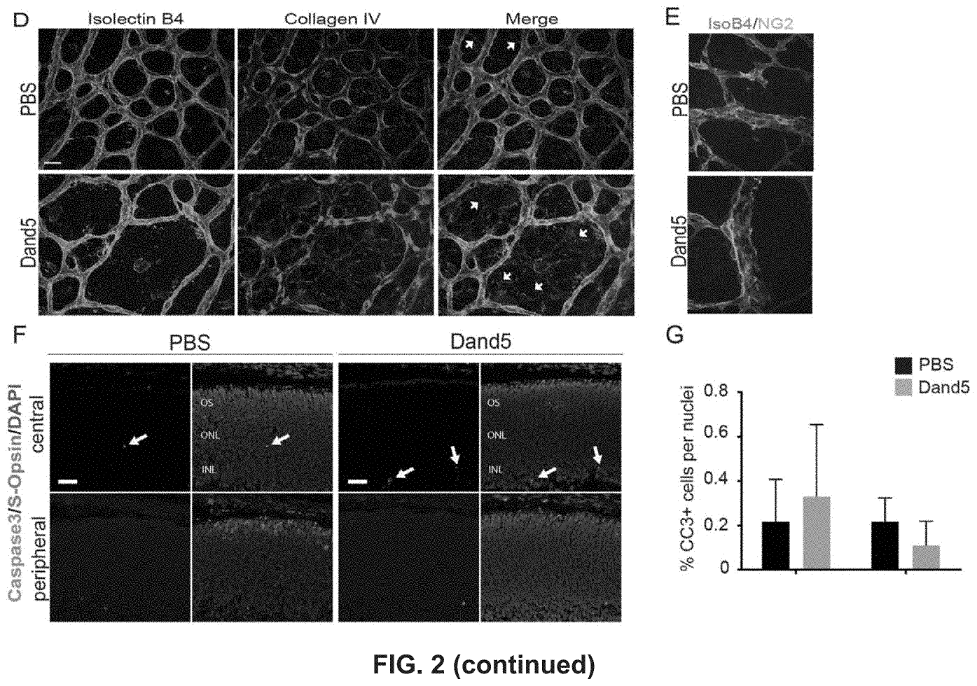

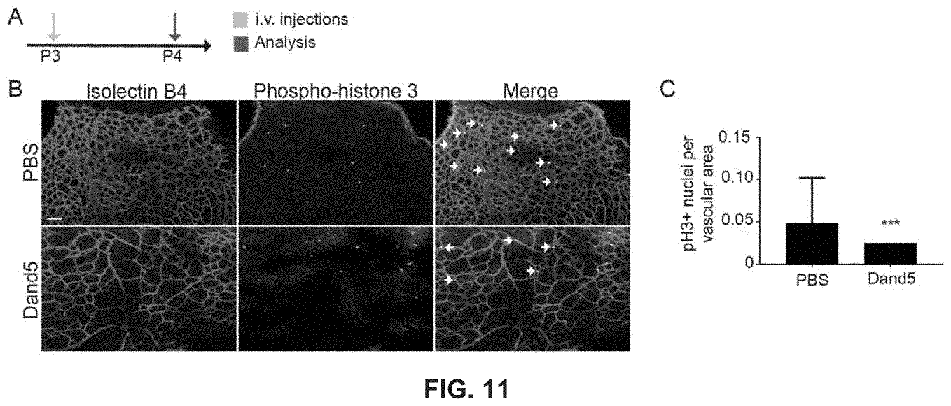

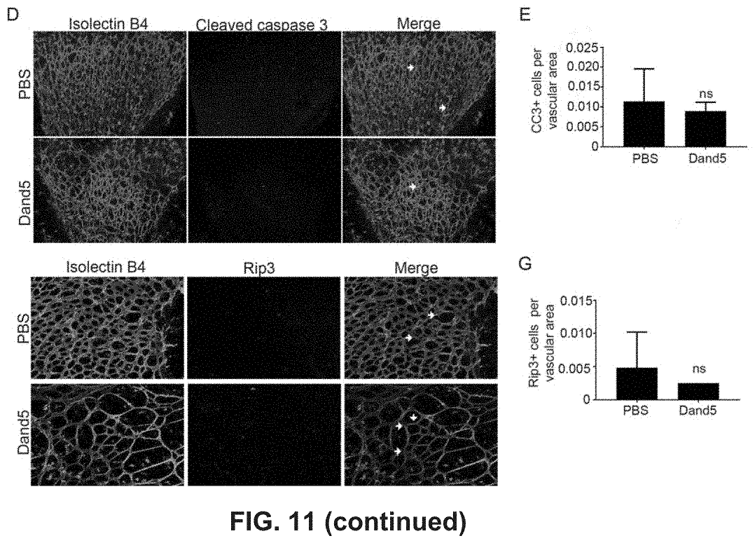

As DAND5 prevents VEGF-induced EC sprouting, proliferation and migration, we evaluated whether it could inhibit retinal vascular development. Newborn mouse pups (P1) received intravitreal injections of recombinant DAND5 (100 ng), and retinas were harvested after 4 days (P5) (FIG. 2A). Delivery of exogenous DAND5 resulted in a dramatic inhibition of blood vessel development. Compared with PBS-injected eyes, a pronounced reduction of vessel area (area covered by vessels; FIG. 2) and microvessel density (ratio of vessel area to vascularized area) was detected in the retinas of DAND5-injected eyes (FIG. 2B, C). The altered vascular pattern was associated with a reduced number of vascular branch points, resulting in a significant reduction of vascular network complexity in DAND5-injected retinas. The inhibition of DAND5 on retinal neovascularization was also significantly more pronounced than that of a VEGF inhibitor (mouse Flt1 Fc) (FIG. 2B, C). The retinal vasculature of DAND5-injected displayed reduced EC proliferation (FIG. 11A-C) but showed no change in apoptosis (FIG. 11D, E) or necroptosis (FIG. 11F-G) consistent with our in vitro data.

Blood vessels constrict in the course of vessel regression and EC retract, leaving behind empty BM sleeves. The retinal vasculature of DAND5-treated eyes had significantly higher numbers of empty type IV collagen (ColIV) BM sleeves (FIG. 2D), suggesting a role of DAND5 in controlling the switch between vessel maintenance and vessel regression. In spite of its effects on endothelial cells and retinal vascular outgrowth, DAND5 injections did not affect retinal pericyte coverage (FIG. 2E) or photoreceptor morphology or apoptosis (FIG. 2F, G).

Acute DAND5 Injection does not Affect Mature Retinal Blood Vessels

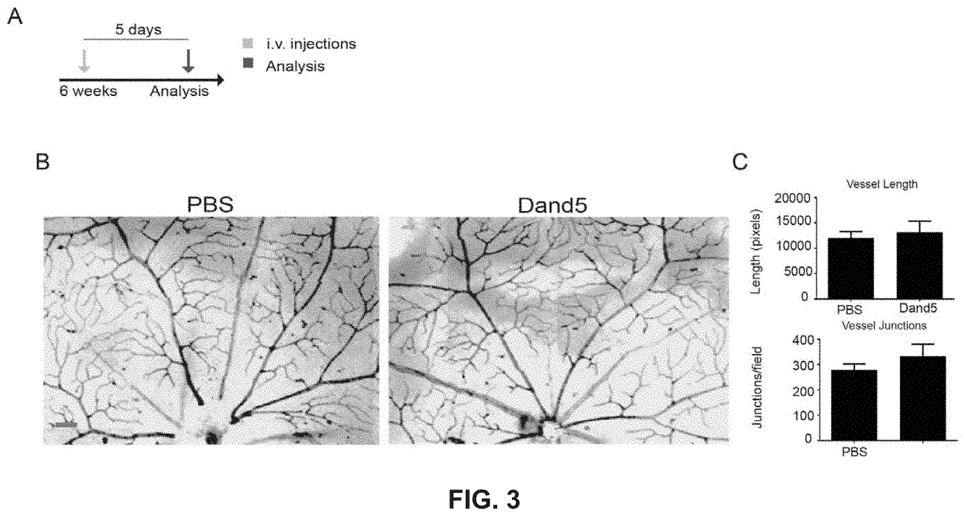

As opposed to newborn pups, which undergo retinal vascular development, a five-day treatment in adult mice (8 week-old) showed no difference in the retinal vasculature between PBS and DAND5-treated eyes (FIG. 3A-C), indicating that DAND5 mediates its effects by preventing the growth of newly formed vessels, rather than inducing the regression of pre-existing vessels. Thus, acute DAND5 injection does not affect mature blood vessels.

Together, these data demonstrate that DAND5 prevents retinal angiogenesis by acting as a regulator of blood vessel stability and EC proliferation, and could be of interest as an inhibitor of angiogenesis in the context of pathological ocular neo-vascularization.

Long-Term Delivery of DAND5 does not Adversely Affect Photoreceptors

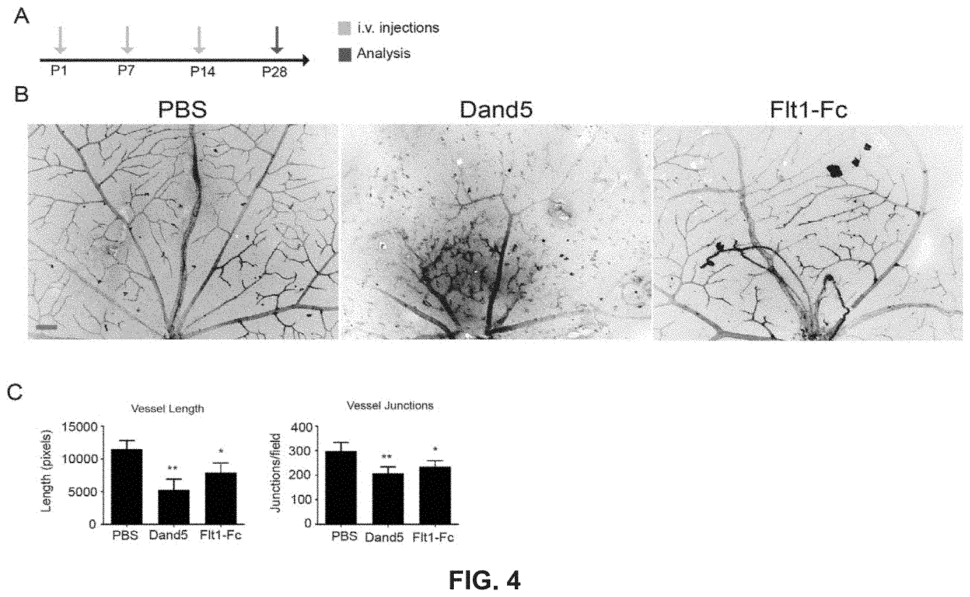

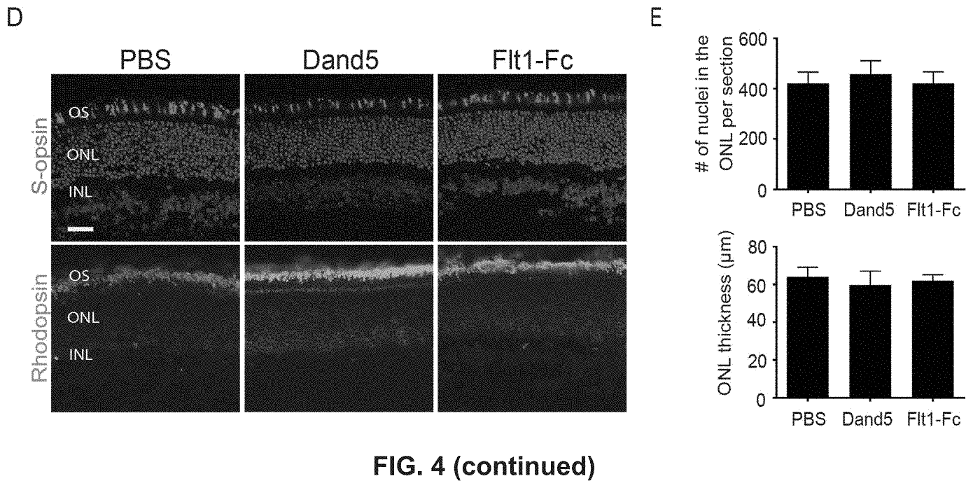

Patients affected by ocular neovascular diseases such as retinopathy of prematurity (ROP) and wet AMD are typically treated with VEGF inhibitors to control pathological angiogenesis. While these agents can significantly reduce retinal and choroidal neovascularization, concerns have been raised regarding their long term safety (3). Particularly, studies have shown that long-term treatment with VEGF inhibitors in mice leads to increased photoreceptor apoptosis and thinning of the neural retina (4). We therefore addressed the long-term effects of DAND5 on photoreceptors and the neural retina. Newborn pups (P1) received repeated injections of DAND5 or Flt1 Fc for 4 weeks (FIG. 4A). While there was a mild decrease in the density of retinal vessels in Flt1Fc-injected animals, a striking reduction in blood vessel formation was observed in the retinas of mice that received DAND5 (FIG. 4B, C). Interestingly, cleaved-caspase 3 staining of photoreceptors did not reveal significant apoptosis in these retinas, showing that DAND5 does not adversely affect photoreceptor (FIG. 4D, E), even though it blocks the development of retinal blood vessels.

DAND5 Inhibits Pathological Retinal and Choroidal Neo-Vascularization

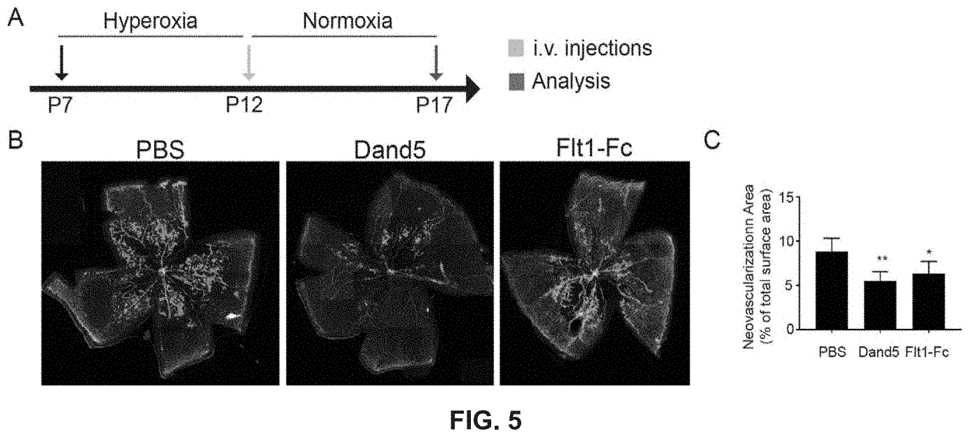

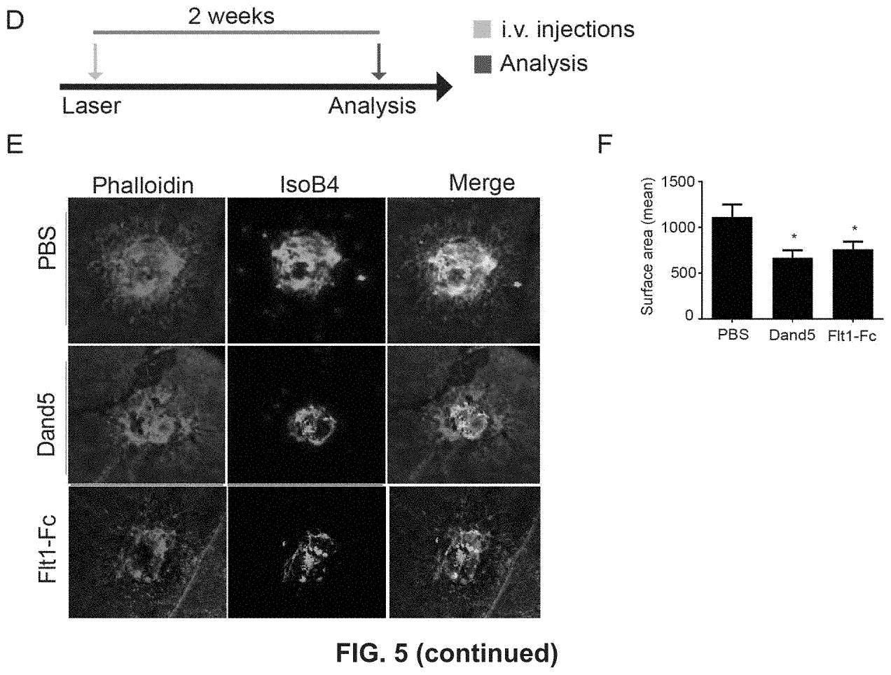

The effects of DAND5 on postnatal developmental angiogenesis led us to evaluate its effects on pathological angiogenesis by subjecting mouse pups to Oxygen-induced Retinopathy (OIR). Briefly, P7 pups were placed in 75% oxygen for 5 days, leading to vaso-obliteration of the retinal vascular plexus. At P12, pups were returned to room air, which stimulates retinal angiogenesis, and leads to the formation of a pathological vascular retinal network characterized by neovascular tufts (FIG. 5A). Treatment of DAND5 at P12 significantly reduced pathological neovascularization in retinas harvested at P17. While revascularization of the central part of the retina was not affected by DAND5, the amount and size of neovascular tufts were significantly reduced in the eyes injected with DAND5 compared to PBS treatment (FIG. 5B, C). As with developmental retinal angiogenesis, DAND5 showed a stronger effect on neovascularization than Flt1 Fc.

The effects of DAND5 on choroidal neovascularization (CNV) were also evaluated by subjecting mice to laser-induced CNV, a model which recapitulates the CNV occurring in wet AMD patients. Briefly, 8-week-old mice subjected to laser impact, followed by intravitreal delivery of either DAND5, Flt1Fc or PBS and CNV was detected 14 days later by staining choroid-sclera whole-mounts with IsoB4 (blood vessels) and phalloidin (RPE) (FIG. 5D). We observed a significant decrease in the area of CNV in mice treated with DAND5 and Flt1 Fc compared with controls (FIG. 5E, F). Together, these observations show that the inhibitory effect of DAND5 on retinal and choroidal neovascularization is significantly better than VEGF inhibition with Flt1Fc.

DAND5 Largely Operates Through a VEGF- and VEGFR-Independent Mechanism

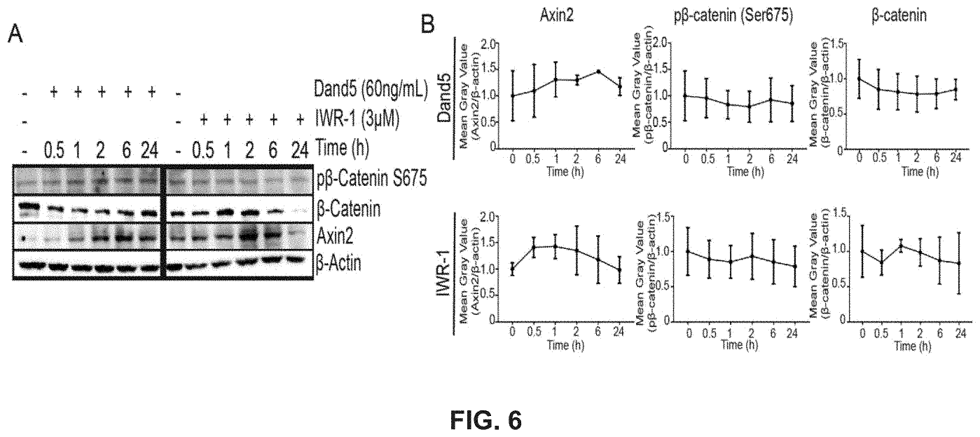

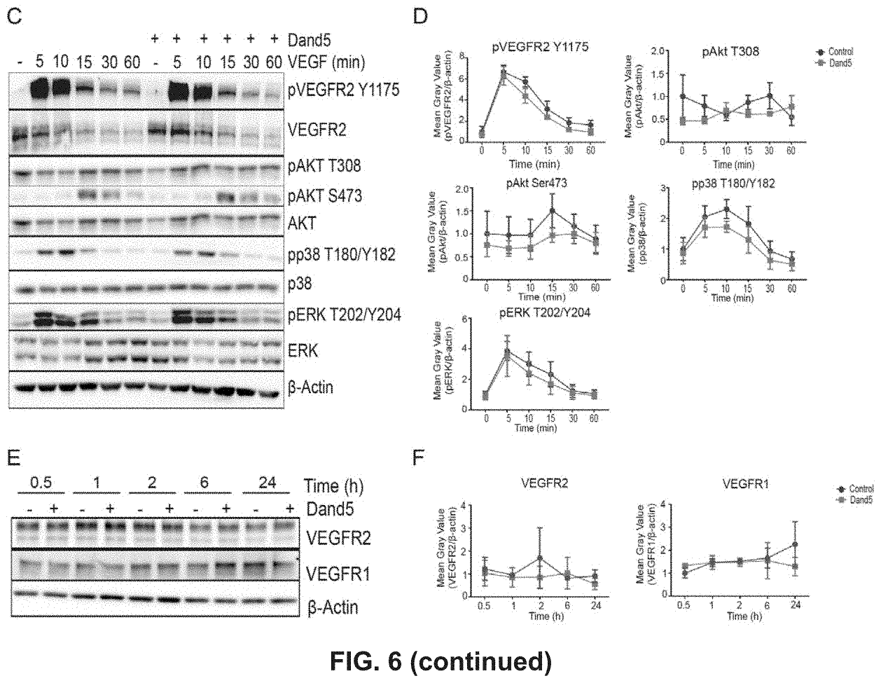

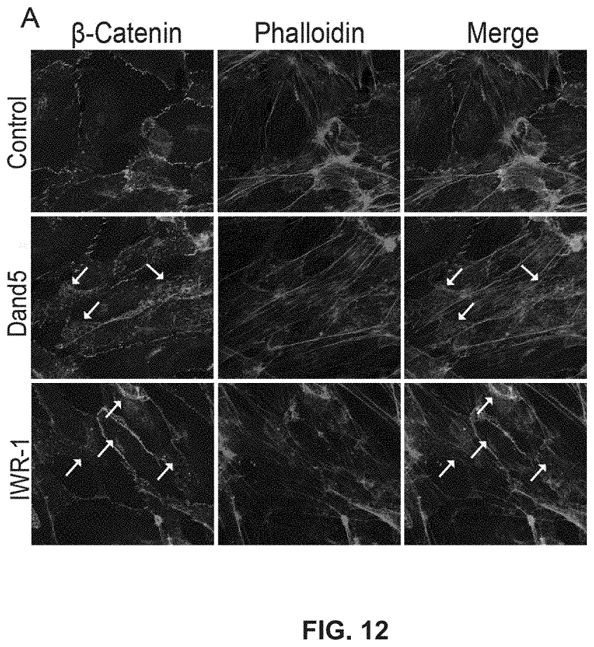

We next evaluated the molecular mechanisms underlying the effects of DAND5 on endothelial cells. DAND5 belongs to the Cerberus family, the members of which have been shown to be extracellular inhibitors of Wnt, BMP and TGFbeta signaling, acting by quenching the activity of ligands of these protein families. Recent reports have demonstrated the important role of Wnt signaling in vascular morphogenesis. Wnt signaling regulates fundamental aspects of vascular development, including cell fate specification, proliferation and survival. Both loss- and gain-of-function experiments of members of the Wnt signaling pathway were found to cause marked alterations of vascular development and endothelial cell specification. As a previous study has shown that DAND5 is a potent inhibitor of Wnt signaling in photoreceptors (5), we assessed whether DAND5 could also modulate Wnt signaling in ECs. HUVECs were treated for up to 24 hours with DAND5 or IWR-1, an inhibitor of Wnt canonical signaling. Protein extracts were harvested and subjected to immunoblotting against downstream effectors of Wnt signaling. HUVECs treated with both DAND5 and IWR-1 showed increased levels of phosphorylated beta-catenin, decreased levels of total beta-catenin and increased levels of Axin2 (FIG. 6A, B), which are associated with decreased Wnt signaling. The levels of β-catenin at different subcellular localizations are regulated by a variety of processes including site-specific phosphorylation of β-catenin. In the absence of an active Wnt signaling, β-catenin localization at cellular junctions is reduced and cytoplasmic β-catenin associates with the destruction complex (6). Staining of HUVECs cultured in the presence of DAND5 showed decreased membrane localization and increased internalization of β-catenin in the cytoplasm (FIG. 12A), which is a precursor event to degradation. Taken together, these data confirm that DAND5 can act as an inhibitor of canonical Wnt signaling in ECs.

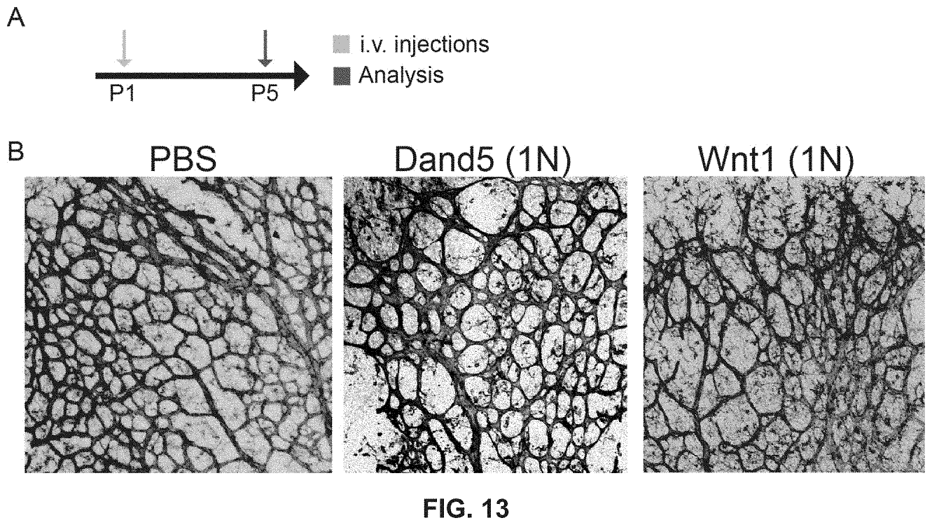

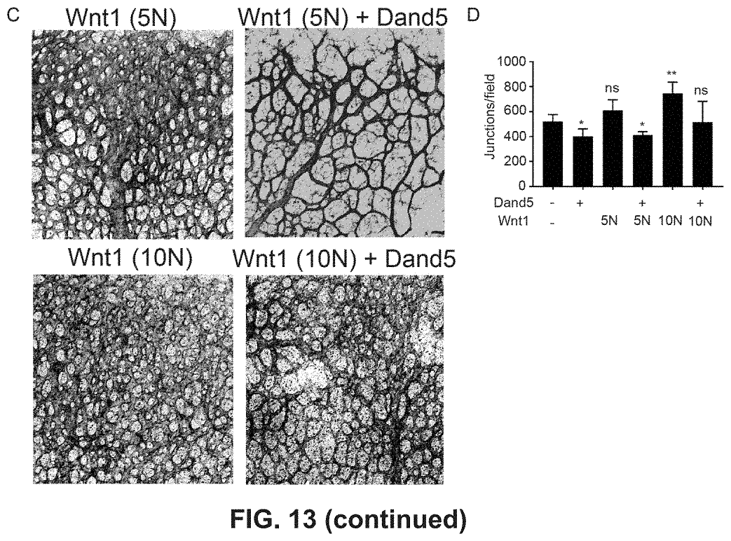

To address the antagonism of DAND5 on Wnt-induced angiogenesis, retinas were injected with recombinant DAND5 (60 ng) with increasing concentrations of Wnt1 (0, 1, 5 or 10× molar ratio) at P1 and retinas were harvested at P5 (FIG. 13A). IsoB4 staining revealed that retinal vascular density was increased following injections of Wnt1, with injections of 16.6 and 33.3 pmoles resulting in significant hypervascularization (FIG. 13B). Concomitants injections of DAND5 with Wnt1 (10 molar ratio) partially rescued the inhibitory effects of DAND5 on retinal angiogenesis, indicating that DAND5 inhibits neovascularization, at least partially, by interfering with Wnt signaling (FIG. 13C, D).

Several studies have demonstrated that the responsiveness of endothelial cells to VEGF can be altered in response to TGFbeta and Wnt stimulation. As our data show that DAND5 can modulate Wnt signaling in endothelial cells, we evaluated whether VEGF signaling was also modulated following DAND5 treatment. HUVECs were starved overnight in the presence or absence of DAND5, followed by VEGF stimulation for up to 60 minutes. Immunoblotting analysis of key signaling events revealed no significant decrease in VEGF-induced phosphorylation of VEGF downstream effectors (FIG. 6C, D). In HUVECs cultured in the presence of DAND5, VEGFR2 phosphorylation at Y1175 was also unaffected (FIG. 6C, D), suggesting that DAND5 operates largely through a VEGF-independent mechanism. Likewise, VEGFR2 and VEGFR1 levels were unaffected upon HUVECs exposure to DAND5 for 0.5-24 h (FIG. 6 E, F).

DAND5 Modulates the Expression of Genes Related to the Mitochondrial Oxidative Metabolism

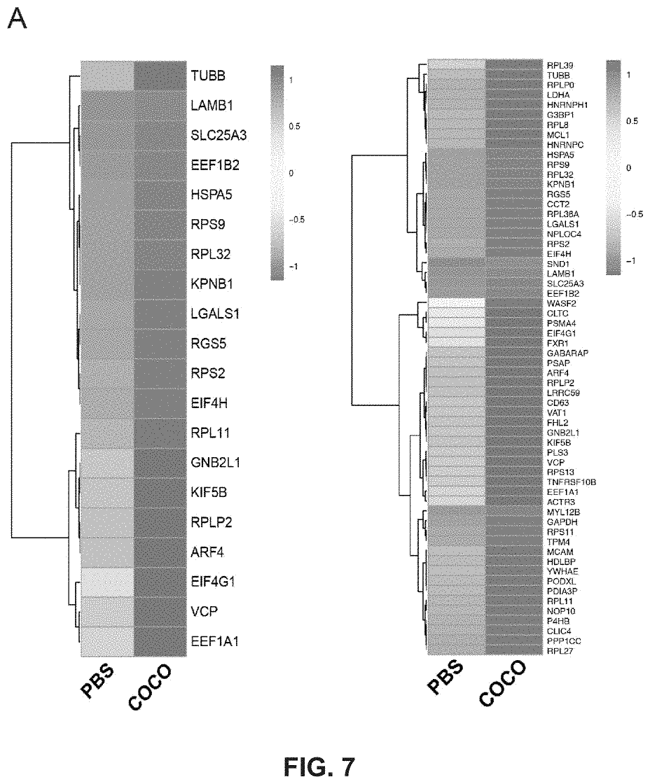

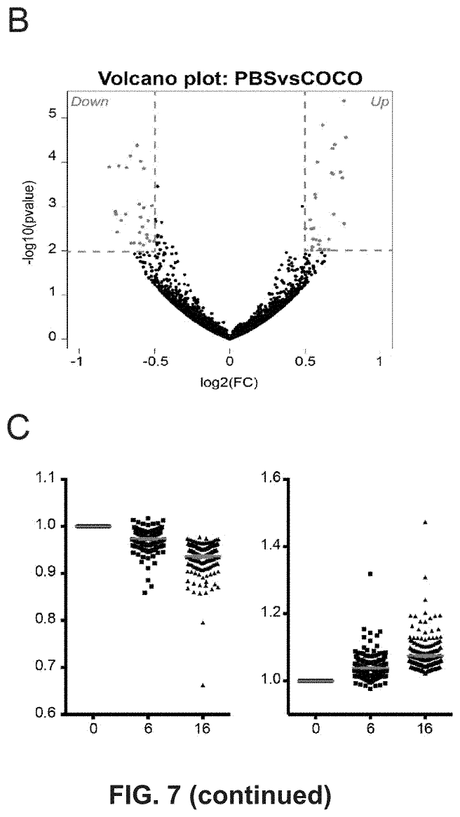

We performed AMPLI-seq analysis of HUVECs exposed or not to recombinant DAND5. This revealed down-regulation of LDHA in DAND5-treated HUVECs (FIG. 7A, B). Lactate de-hydrogenase A (LDHA) regulates lactate metabolism during anaerobic respiration. LDHA catalyzes the conversion of L-Lactate and NAD into pyruvate and NADH, altogether suggesting a possible impact of DAND5 on the mitochondrial metabolism.

DAND5 Modulates the Mitochondrial Oxidative Metabolism in Endothelial Cells

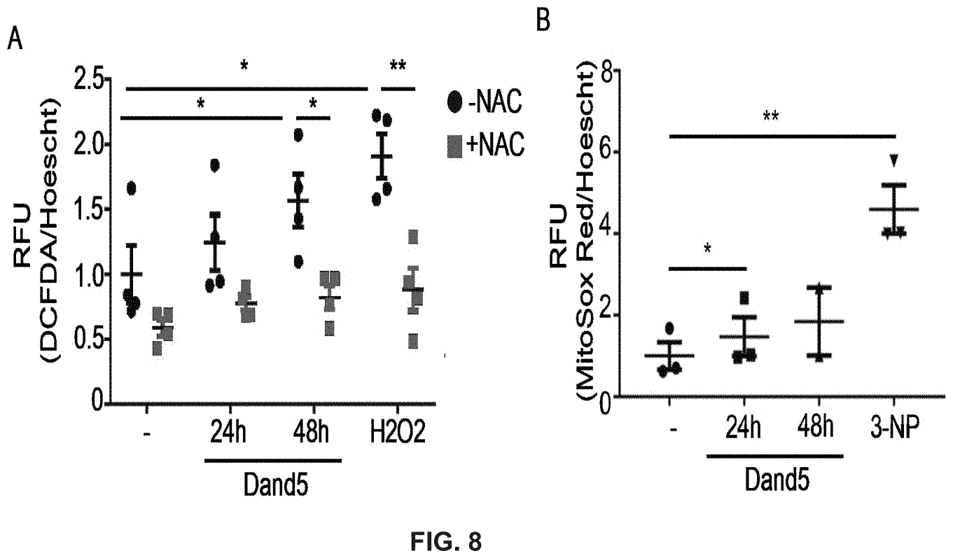

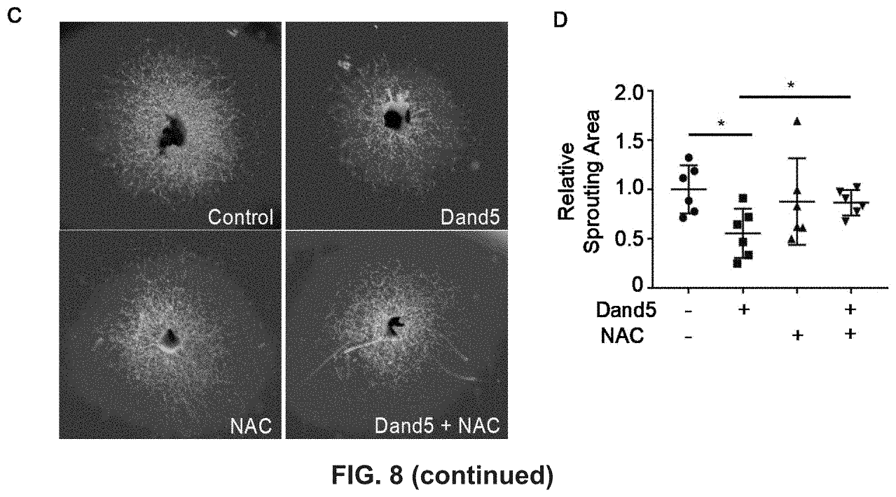

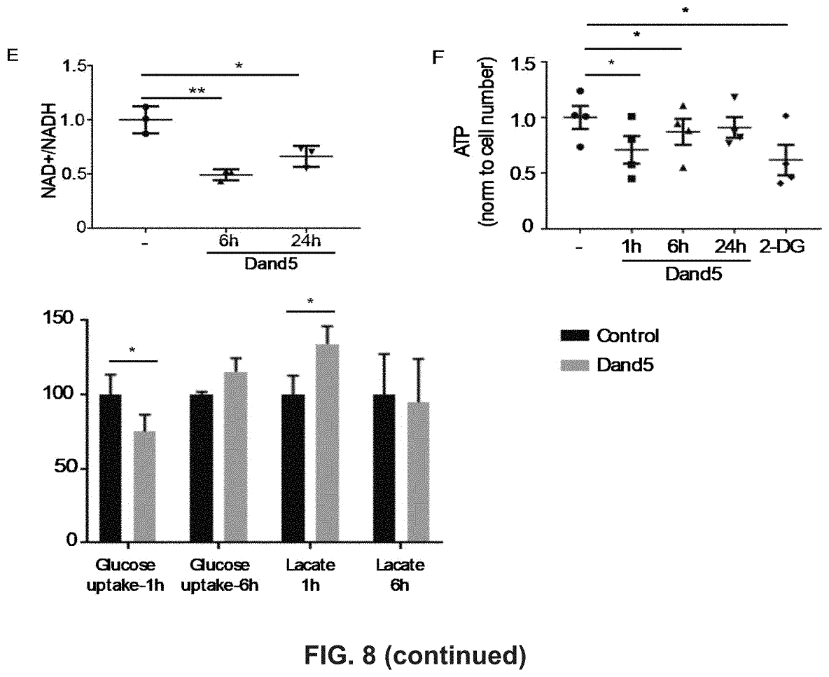

We exposed or not HUVECs to recombinant DAND5 and measured the concentration of free radicals using DCFDA and MitoSoxRed™. We found that DAND5 induced elevation of free radicals 24 h and 48 h post-treatment, and that this could be overcome by exposing cells to N-acetyl cystein (NAC), a potent free radical scavenger (FIG. 8A). Notably, time-course analysis revealed that mitochondrial reactive oxygen species (ROS), as detected using MitoSoxRed™, were induced before cytosolic ROS upon DAND5 treatment, suggesting a direct effect on mitochondria (FIG. 8B and not shown). Accordingly, the growth of mouse choroidal explants treated with DAND5 was partially rescued when also treated with NAC (FIG. 8C-D). To further test this, we measure the NAD+/NADH ratio in cells treated or not with DAND5. We found that the NAD+/NADH ratio was decreased following exposure of HUVECs to DAND5 (FIG. 8E). Likewise, glucose uptake was reduced and lactate levels were increased in DAND5-treated HUVECs after 1 h (FIG. 8E, lower panel). A last, we measure that ATP levels were reduced in HUVECs after 1 h of treatment with DAND5 (FIG. 8F), altogether suggesting a direct and rapid effect of DAND5 on the mitochondrial metabolism in HUVECs.

DAND5 Localizes to Mitochondria in HUVECs

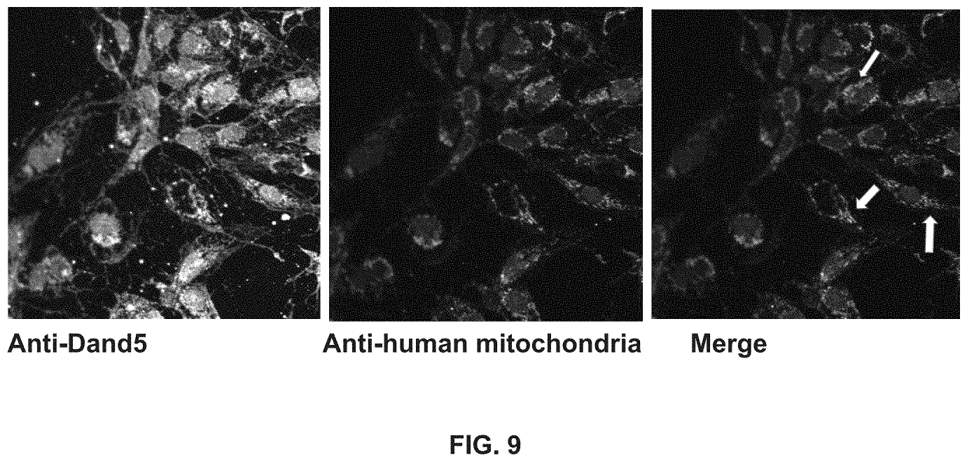

To test where DAND5 is localizes in treated HUVECs, we exposed HUVECs to human recombinant DAND5 for 1 h and performed immune-fluorescence using human anti-mitochondrial antigen antibody and a human anti-DAND5 antibody. This revealed that DAND5 was not detectable in untreated HUVECs (not shown). However, in HUVECs exposed to DAND5, we observed robust co-localization of DAND5 with mitochondria (FIG. 9), suggesting that DAND5 is rapidly transported from the extra-cellular milieu to the mitochondria, most likely by active transport of endocytic vesicles.

DAND5 is a Candidate Therapeutic Molecule for Retinal Transplantation Therapy

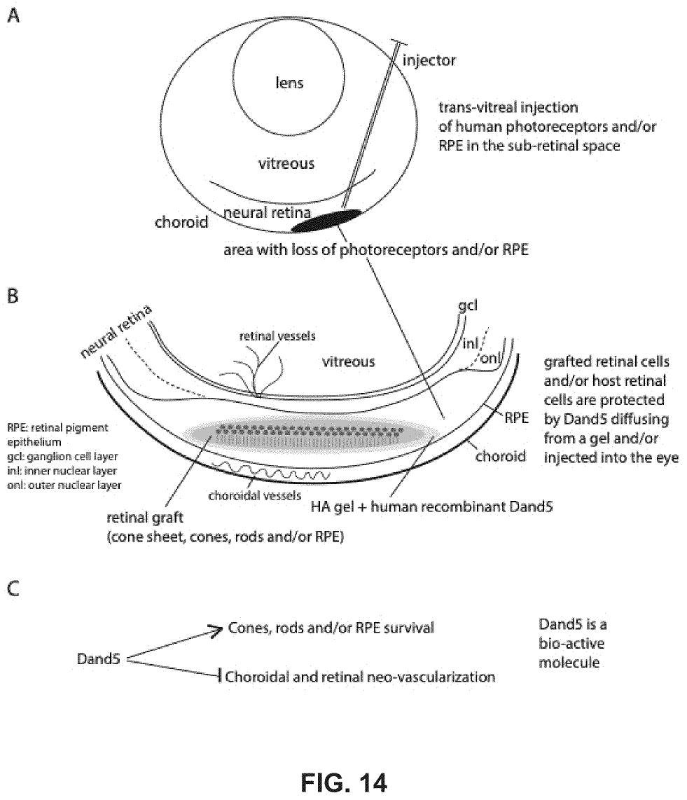

In retinal degenerative diseases such as wet and dry AMD, as well as in cone and cone/rod dystrophies and Stargardt's disease, cone photoreceptors and RPE degenerate, leading to loss of central (macular) vision and legal blindness. Loss of central vision is also present in end-stage retinitis pigmentosa. Cell replacement transplantation therapy using photoreceptors and/or RPE is a possible treatment for these blinding diseases. Yet, local inflammation, neo-vascularization and surgical stress are common problems that can be observed in these pathological conditions, which are altogether predicted to be detrimental to the survival and integration of grafted retinal cells. Our newly presented results suggest that DAND5 is a potent therapeutic to prevent or reverse pathological neo-vascularization. Likewise, direct and indirect evidences suggest that DAND5 can promote pluripotent stem cell differentiation into cone photoreceptors and that DAND5 acts as a neurotrophic factor for photoreceptors and RPE. Hence, we propose that this dual biological activity of DAND5 can be exploited in retinal cell transplantation therapy as to prevent retinal and/or choroidal neo-vascularization and promote grafted photoreceptors and/or RPE survival (FIG. 14A-C). The neurotrophic activity of DAND5 may be also beneficial for endogenous, host retinal cells.

### DISCUSSION

Current therapies to treat neo-vascular ophthalmic diseases are mostly centered on the inhibition of a single factor, VEGF. The outcomes of anti-VEGF treatments are to counteract pathological neovascularization and disease progression, to arrest visual impairment and, in the best case, to gain the recovery of vision. Some molecules targeting VEGF are currently used in ophthalmology, and many more are under investigation in clinical trials for either AMD, ROP, or other eye diseases characterized by neovascularization. While VEGF blocking agents have provided significant clinical benefits, a number of patients show poor responses to these drugs and some concerns have been raised regarding the long-term use of VEGF inhibitors. It has been proposed that pan-VEGF blockade is responsible of increasing geographic atrophy in AMD patients, a gradual complication characterized, among others, by choriocapillaries and RPE atrophy, photoreceptors death, and leading to a progressive visual loss. It is therefore of great clinical interest to identify novel targets that could complement or replace current treatments.

In the present example we demonstrate that DAND5, a secreted antagonist of BMP, TGFbeta, and Wnt signaling molecules, is a potent inhibitor of neovascularization in the eye. Injection of DAND5 during developmental retinal angiogenesis severely delayed the development of new blood vessels. Interestingly, the effect of DAND5 on blood vessel formation appeared to be more potent than that of a commonly used VEGF inhibitor, Flt1Fc. In experimental models of choroidal neovascularization and vascular retinopathy, DAND5 also displayed potent inhibitory effects on neovascularization. Importantly, DAND5 proved to act specifically on developing blood vessels, as intravitreal delivery in adult mice did not result in any significant effects on the mature vasculature.

While VEGF inhibitors have shown good clinical efficacy for the prevention of neovascularization in wet AMD, they also carry significant risks of unwanted side effects in non-vascular cells in the eye. A study by Saint-Geniez et al., has shown that long-term administration of a VEGF trap (Flt1Fc) induces photoreceptor apoptosis and loss, resulting in decreased retinal function. Furthermore, recent trials have also shown that in humans, long-term treatments with VEGF inhibitors such as ranibizumab or bevacizumab result in increased risks of geographic atrophy (GA) progression. While there is still ongoing research on the mechanism of the cause of GA, one of the reasons for GA could be due to the drastic reduction of VEGF from anti-VEGF therapy. It was found that RPE-derived VEGF is necessary for maintenance of choriocapillaris and the absence of specific VEGF isoforms results in retinal degeneration, therefore highlighting the need of identifying angiogenesis inhibitors that target alternative signaling pathways. Furthermore, photoreceptors have been shown to express VEGFR2, and specific deletion of this receptor in cone photoreceptors in mice has been shown to result in decreased photoreceptor density and morphological anomalies, characterized by shorter appearance and disordered arrangement. The present data show that long-term delivery of DAND5, while having a potent effect on newly-formed blood vessels, does not adversely affect photoreceptors. A recently published study showed that DAND5 is endogenously expressed in the retina, and that it is required for the maintenance of cultured photoreceptors (16). DAND5-based therapies could therefore be used for long-term inhibition of neovascularization in ocular pathologies without the risks associated with VEGF inhibitors. Alternatively, DAND5 may be used in combination with anti-VEGF to improve response in “resistant” patients or to reduce anti-VEGF dosage and/or treatment frequency in others, thus potentially mitigating side effects.

Mechanistically, our data show that DAND5 prevents neovascularization at least in part by inhibiting Wnt signaling. Several recently published studies have shown that inhibition of both canonical and non-canonical Wnt signaling in retinal vessels leads to hypo-vascularization. Endothelial cell migration and proliferation are essential steps in angiogenesis and are regulated by Wnt signaling. In human retinal vascular diseases, mutations in the Fz4 (Wnt receptor) and LRP5 (Wnt coreceptor) genes have also been found to associate with abnormal impaired angiogenesis and specific inhibitors of these pathways will inhibit vessel growth.

More importantly, we have found that DAND5 rapidly affects the mitochondrial oxidative metabolism in vascular endothelial cells, and this in a VEGF-, VEGFR1- and VEGFR2-independent manner. DAND5 may be thus a suitable alternative or complement to anti-VEGF for the treatment of neo-vascular diseases affecting the eye. Since retinal degenerative diseases such as wet AMD are also frequently associated with loss of cone photoreceptors and RPE, there is a need for cell replacement therapies in order to restore lost vision. However, limitations such as cell death of the graft owing to the lack of neurotrophic support, local neuro-inflammation and neo-vascularization exist. Hence, local (ocular) neuro-inflammation is thought to drive retinal and choroidal neo-vascularization. To overcome these problems, we have proposed to add DAND5, which shows anti-angiogenic and pro-neurotrophic activity, with grafted photoreceptors and/or RPE. The human recombinant DAND5 molecule could be released from a gel and/or beads surrounding the graft (or grafted cells) to allow short-term and medium-term delivery at the site of the graft. In complementation, DAND5 could be injected directly into the vitreous at the time of surgery or after to deliver its therapeutic activity.

Taken together, we thus demonstrated that DAND5 is a potent inhibitor of physiological and pathological retinal angiogenesis acting independently of VEGF signaling.

### REFERENCES

1. Marneros, A. G., NLRP3 inflammasome blockade inhibits VEGF-A-induced age-related macular degeneration. Cell Rep, 2013. 4(5): p. 945-58.
2. Holz, F. G., S. Schmitz-Valckenberg, and M. Fleckenstein, Recent developments in the treatment of age-related macular degeneration. The Journal of clinical investigation, 2014. 124(4): p. 1430-1438.
3. Ferris, F. L., et al., AGe-related macular degeneration and blindness due to neovascular maculopathy. Archives of Ophthalmology, 1984. 102(11): p. 1640-1642.
4. Kwak, N., et al., VEGF is major stimulator in model of choroidal neovascularization. Investigative ophthalmology & visual science, 2000. 41(10): p. 3158-3164.
5. Miller, J. W., Age-Related Macular Degeneration Revisited—Piecing the Puzzle: The LXIX Edward Jackson Memorial Lecture. American Journal of Ophthalmology, 2013. 155(1): p. 1-35.e13.
6. Yadav, L., et al., Tumour Angiogenesis and Angiogenic Inhibitors: A Review. J Clin Diagn Res, 2015. 9(6): p. Xe01-xe05.
7. Mazure, N. M., et al., Induction of vascular endothelial growth factor by hypoxia is modulated by a phosphatidylinositol 3-kinase/Akt signaling pathway in Ha-ras-transformed cells through a hypoxia inducible factor-1 transcriptional element. Blood, 1997. 90(9): p. 3322-31.
8. Leung, D. W., et al., Vascular endothelial growth factor is a secreted angiogenic mitogen. Science, 1989. 246(4935): p. 1306-9.
9. Zachary, I., Integration of mitogenic and migratory signals from G-Proteincoupled receptor and tyrosine kinases. Biochemical Society Transactions, 2003. 31(part 6).
10. Smith, G. A., et al., The cellular response to vascular endothelial growth factors requires co-ordinated signal transduction, trafficking and proteolysis. Biosci Rep, 2015.
11. Houck, K. A., et al., The vascular endothelial growth factor family: identification of a fourth molecular species and characterization of alternative splicing of RNA. Mol Endocrinol, 1991. 5(12): p. 1806-14.
12. Kumar, V., et al., Robbins and Cotran Pathologic Basis of Disease. 2014: Elsevier Science Health Science Division.
13. Saint-Geniez, M., et al., Endogenous VEGF Is Required for Visual Function: Evidence for a Survival Role on Müller Cells and Photoreceptors. PLoS ONE, 2008. 3(11): p. e3554.
14. Bell, E., et al., Cell fate specification and competence by DAND5, a maternal BMP, TGFbeta and Wnt inhibitor. Development, 2003. 130(7): p. 1381-9.
15. Clifford, R. L., K. Deacon, and A. J. Knox, Novel regulation of vascular endothelialgrowth factor-A (VEGF-A) by transforming growth factor (beta)1: requirement for Smads, (beta)-CATENIN, AND GSK3(beta). J Biol Chem, 2008. 283(51): p. 35337-53.
16. Zhou, S., Flamier, A., Abdouh, M., Tétreault, N., Barabino, A., Wadhwa, S., and Bernier, G. (2015). Differentiation of human embryonic stem cells into cone photoreceptors through simultaneous inhibition of BMP, TGFβ and Wnt signaling. Development 142, 3294-3306.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a DAN family BMP antagonist” includes one or more of such antagonists and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A method for treating an ocular condition, comprising administering to a mammalian subject in need thereof an effective amount of a DAN family BMP antagonist.

2. The method of claim 1, wherein said a DAN family BMP antagonist is DAND5.

3. The method of claim 1, wherein said administering comprises intravitreal injection and/or subretinal injection.

4. The method of claim 1, wherein said ocular condition is selected from the group consisting of: wet age-related macular degeneration (AMD), dry AMD, retinopathy of prematurity, diabetic retinopathy, ocular neovascularization, Stargardt's disease, inherited retinal dystrophy, cone and cone/rod dystrophy, retinitis pigmentosa, ocular angiogenesis, photoreceptors damage, retinal pigment epithelial cells (RPEC) detachment, and other retinal degenerative diseases affecting photoreceptors.

5. The method of claim 1, wherein said ocular condition is selected from the group consisting of: wet age-related macular degeneration (AMD), retinopathy of prematurity and diabetic retinopathy.

6. The method of claim 1, wherein said DAN family BMP antagonist comprises at least one, preferably two or more, of the following biological activities:
i) suppress VEGF-induced tube formation of human umbilical vein endothelial cells (HUVECs);
ii) prevents VEGF-induced cell migration of HUVECs;
iii) inhibits sprouting, migration and/or cellular proliferation of HUVECs;
iv) inhibits retinal blood vessel development;
v) reduction of HUVECs proliferation without causing apoptosis or necroptosis;
vi) inhibits retinal neovascularization;
vii) reduces pathological neovascularization in retina;
viii) prevents choroidal neovascularization (CNV) in a mice model of laser-induced CNV;
ix) inhibits Wnt signaling in HUVECs;
x) inhibits Wnt neo-vascular effect in the mouse retina;
xi) induces elevation of free radicals in HUVECs;
xii) affects mitochondrial oxidative metabolism to decrease the NAD+/NADH ratio and ATP production;
xiii) blocks Glucose uptake and increases Lactate production in HUVECs;
xiv) localizes to mitochondria in treated HUVECs; and
xv) promote differentiation and survival of human photoreceptors and retinal pigment epithelium (RPE).

7-18. (canceled)

19. A method for inhibiting and/or preventing ocular neovascularization and/or ocular angiogenesis in a mammalian subject, the method comprising administering to a mammalian subject in need thereof an effective amount of a DAN family BMP antagonist.

20. The method of claim 19, wherein said DAN family BMP antagonist is DAND5.

21. The method of claim 19, wherein said administering comprises intravitreal injection and/or subretinal injection.

22. The method of claim 19, for inhibiting and/or preventing choroidal neovascularization and retinal neovascularization.

23. (canceled)

24. A pharmaceutical composition for treating an ocular condition, said pharmaceutical composition comprising a DAN family BMP antagonist, and a pharmaceutically acceptable carrier or excipient.

25-29. (canceled)

30. The method of claim 1, wherein said treating comprises administering retinal cells to the mammalian subject, and wherein said administering comprises at least one of: (i) coating said retinal cells with a DAN family BMP antagonist; (ii) incorporating said retinal cells in a matrix comprising a DAN family BMP antagonist; and (iii) injecting a DAN family BMP antagonist into the vitreous and/or subretinal space at the time of the retinal cell transplantation and/or shortly thereafter.

31-39. (canceled)

40. The pharmaceutical composition of claim 24, wherein said DAN family BMP antagonist is DAND5.

41. The pharmaceutical composition of claim 24, wherein said ocular condition is selected from the group consisting of: wet age-related macular degeneration (AMD), dry AMD, retinopathy of prematurity, diabetic retinopathy, ocular neovascularization, Stargardt's disease, inherited retinal dystrophy, cone and cone/rod dystrophy, retinitis pigmentosa, ocular angiogenesis, photoreceptors damage, retinal pigment epithelial cells (RPEC) detachment, and other retinal degenerative diseases affecting photoreceptors.

42. The pharmaceutical composition of claim 24, wherein said ocular condition is selected from the group consisting of: wet age-related macular degeneration (AMD), retinopathy of prematurity and diabetic retinopathy.

43. The pharmaceutical composition of claim 24, wherein said DAN family BMP antagonist comprises at least one, preferably two or more, of the following biological activities:
i) suppress VEGF-induced tube formation of human umbilical vein endothelial cells (HUVECs);
ii) prevents VEGF-induced cell migration of HUVECs;
iii) inhibits sprouting, migration and/or cellular proliferation of HUVECs;
iv) inhibits retinal blood vessel development;
v) reduction of HUVECs proliferation without causing apoptosis or necroptosis;
vi) inhibits retinal neovascularization;
vii) reduces pathological neovascularization in retina;
viii) prevents choroidal neovascularization (CNV) in a mice model of laser-induced CNV;
ix) inhibits Wnt signaling in HUVECs;
x) inhibits Wnt neo-vascular effect in the mouse retina;
xi) induces elevation of free radicals in HUVECs;
xii) affects mitochondrial oxidative metabolism to decrease the NAD+/NADH ratio and ATP production;
xiii) blocks Glucose uptake and increases Lactate production in HUVECs;
xiv) localizes to mitochondria in treated HUVECs; and
xv) promote differentiation and survival of human photoreceptors and retinal pigment epithelium (RPE).

44. The pharmaceutical composition of claim 24, wherein said composition is for inhibiting and/or preventing at least one of choroidal neovascularization and retinal neovascularization.

45. The pharmaceutical composition of claim 24, wherein said composition is formulated for injection into the vitreous and/or subretinal space.
